(19) United States
(12) Patent Application Publication
(10) Pub. No.: US 2016/0091489 A1
(43) Pub. Date: Mar. 31, 2016

(54) DEVICES AND METHODS FOR ISOLATING CELLS

(71) Applicant: UNIVERSITY OF FLORIDA RESEARCH FOUNDATION, INCORPORATED, Gainesville, FL (US)

(72) Inventors: ZHONGHUI HUGH FAN, GAINESVILLE, FL (US); WEIAN SHENG, MILLBRAE, CA (US); TAO CHEN, MILLBRAE, CA (US); WEIHONG TAN, GAINESVILLE, FL (US)

(21) Appl. No.: 14/890,918

(22) PCT Filed: Jun. 3, 2014
(86) PCT No.: PCT/US2014/040649

§ 371 Date: Nov. 13, 2015

Related U.S. Application Data
(60) Provisional application No. 61/830,356, filed on Jun. 3, 2013.

(51) Int. Cl. G01N 33/543

(52) U.S. Cl. CPC G01N 33/54353; G01N 33/54386

(57) ABSTRACT

The subject invention pertains to devices and methods of isolating target cells from a population of cells. The devices comprise of one or more microfluidic channels and scaffolding particles conjugated with one or more ligands that bind to the target cells. The scaffolding particles with one or more ligands are attached on to the surface of the one or more microfluidic channels. The methods of the current invention comprise passing the population of cells through the microfluidic channels of the devices of the current invention to facilitate interaction and capture of the target cells by the scaffolding particles-ligand conjugates, washing the device by a washing solution to remove the cells non-specifically bound to the scaffolding particle-ligand conjugates, releasing the captured target cells from the scaffolding particle-ligand conjugates, and collecting the released target cells.

### CROSS-REFERENCE TO RELATED APPLICATION

This application claims the benefit of U.S. Provisional Application Ser. No. 61/830,356, filed June 3, 2013, the disclosure of which is hereby incorporated by reference in its entirety, including all figures, tables and amino acid or nucleic acid sequences.

This invention was made with government support under K25CA149080 awarded by National Institutes of Health (NIH). The government has certain rights in the invention.

### BACKGROUND OF THE INVENTION

Isolation of target cells, i.e. the cells of interest, from a population of cells should be specific, efficient, and quick. Specificity of the isolation technique ensures that all or most of the isolated cells are the target cells. Efficiency of the isolation technique ensures that all or most of the target cells present in the initial population of cells are isolated. Quick isolation of cells ensures that the isolated cells are viable and in good health during and after isolation procedures.

Isolation of target cells based on specific cell surface molecules is routinely performed. For example, cells having specific cell surface protein receptors can be isolated using ligands that specifically bind to the cell surface protein. Microfluidic devices with monovalent capture ligands, including antibodies¹¹⁻¹⁵ and nucleic acid aptamers,¹⁶⁻¹⁸ have been used for immunocapture of rare tumor cells. However, most efforts for enhancing ligand-cell interactions and increasing the sensitivity of cell capture are based on engineering complicated structures inside the microfluidic devices, such as microposts, sinusoidal channels, and silicon nanopillars, etc.¹⁹⁻²² These structures make the device fabrication time-consuming and also induce significant nonspecific cell capture causing low specificity.

Multivalent binding, which is the simultaneous interaction of multiple ligands on one entity with the complementary receptors on another, has been widely used for achieving high-affinity molecular recognition.²³⁻²⁷ Multivalent binding between ligands and targets in biological samples has also been investigated.²⁸⁻³⁰ For example, scaffolds from numerous nanoscale structures, such as dendrimers,³¹⁻³²nanorods,³³ nanoparticles,³⁴ polymers³⁵ and proteins, have been used for assembling multiple ligands on scaffolding particles to achieve multivalent binding. Recently, nucleic acid aptamers have been selected for targeting numerous cancers³⁶⁻³⁷ and nanomaterial-aptamer conjugates have been extensively used for enhanced molecular recognition.

Isolation of Circulating Tumor Cells (CTCs) from a subject is of great interest and various techniques of isolating these target cells are practiced. CTCs are cancer cells shed from either primary tumors or metastatic sites and are related to initiation of metastasis and spread of cancer to distant organs. Thus studying CTCs hold the key for understanding metastasis, diagnosing cancer, and monitoring treatment response.⁴⁻⁶ However, the extraordinary rarity of CTCs makes their isolation and characterization challenging. Traditionally, methods based on flow cytometry have been used in clinics, but with considerable false negatives, i.e. low specificity, and low detection sensitivity, i.e. low efficiency.⁷⁻⁸ The only FDA-approved CTC enumeration method is CellSearch Assay, which uses antibody-coated magnetic beads for CTC isolation. However, it also suffers from low CTC-capture efficiency.⁹⁻¹⁰

Various aspects and embodiments of this invention provide microfluidic devices which capture target cells from a population of cells. The devices and methods of the current invention can be used for isolation of CTCs from peripheral blood. ¹⁻³ The current invention utilizes nanoparticle based multivalent binding to isolate cells using microfluidic devices. DNA nanospheres can be produced by conjugating nanoparticles, for example, gold nanoparticles (AuNPs), with multiple ligands having binding affinity for different sites on the target cell surface, for example, DNA aptamers having different binding affinity. These nanospheres can be used as multivalent scaffolding particles to isolate target cells.^{42, 43} Thus, nanoparticle-based multivalent binding can be used for capturing target cells from a population of cells with high efficiency and specificity at increased flow rate and high sample throughput.

### BRIEF SUMMARY OF THE INVENTION

Devices and methods for isolating target cells from a population of cells in efficient, specific, fast, and high throughput manner are provided. Devices, as disclosed herein, may comprise of one or more microfluidic channels and scaffolding particles conjugated with one or more ligands that bind to the target cells, wherein the scaffolding particles with one or more ligands are attached on to the surface of the one or more microfluidic channels. In certain embodiments, devices and methods for isolating CTCs from peripheral blood, the devices comprising gold nanoparticles conjugated with a plurality of DNA aptamers attached to the inner surface of microfluidic channels, wherein the DNA aptamers bind to and capture CTCs from the blood by specifically binding to different sites on the surface of the CTCs.

Methods for isolation of target cells from a population of cells are also provided, the methods comprising passing the population of cells through the microfluidic devices to facilitate interaction and capture of the target cells by the scaffolding particles-ligand conjugates, washing the scaffolding particles-ligand conjugates with a washing solution to remove the cells non-specifically bound to the scaffolding particle-ligand conjugates, releasing the captured target cells from the scaffolding particle-ligand conjugates, and collecting the released target cells.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIGS. 1A-1E. a-b) Illustration of enhanced cell capture using AuNP-aptamer modified surface. With AuNP conjugation (a), multiple aptamers on the AuNP surfaces bind with multiple receptors on the cell membrane, leading to cooperative, multivalent interactions; Without AuNP (b), aptamer alone binds with receptors via monovalent interaction, with much less interactions. c-d) Transmission electron microscopy (TEM) image of AuNPs (c), and AuNPs conjugated with aptamers (d), scale bar=100 nm. e) Comparison between AuNP and AuNP-aptamer in terms of particle diameters from TEM images, hydrodynamic diameters from dynamic light scattering (DLS) measurements, and zeta-potential measurements.

FIGS. 2A-2D. Flow cytometry shows the strong and specific binding of AuNP-sgc8 aptamer conjugates (AuNP-sgc8) with target CEM cells. a) CEM cells selectively bind with free sgc8 and AuNP-sgc8 aptamers; an enhanced binding with AuNP-aptamer conjugates than aptamers alone was observed, even with 10x lower concentration. b) Control Ramos cells did not bind with either AuNP-sgc8 or sgc8 alone (with no signal shift for either case), demonstrating the specificity of free sgc8 and AuNP-sgc8 aptamers to CEM cells. c-d) Flow cytometry analysis determines the binding affinity of AuNP-sgc8 (c) and sgc8 alone (d) to CEM cells.

FIGS. 3A-3D. a-b) Representative image of the target CEM cells (red) and control Ramos cells (blue) captured in the flat channel device using (a) AuNP-sgc8 aptamer conjugates; (b) sgc8 aptamer alone. c) Comparison of CEM cell capture efficiency in PBS between AuNP-aptamer and aptamer alone when they were coated in a flat channel device, at flow rates from 0.4 μL/s to 2.4 μL/s. d) Comparison of the capture purity of target CEM cells between AuNP-aptamer and aptamer alone; no significant difference was observed. Error bars represent standard deviations (n=3).

FIGS. 4A-4D. a-b) Spatial distribution of surface-captured CEM cells along the 50 mm-long microchannel in the flat channel device at different flow rates of (a) 1.2 μL/s and (b) 2.4 μL/s; c) Capture efficiency for 100,000, 10,000, 1000 and 100 CEM cells spiked in 1 mL of lysed blood; d) CEM cell capture efficiency from lysed blood or whole blood at the same flow rate (1.2 μL/s). Error bars represent the standard deviations of triplicate experiments.

FIGS. 5A-5D. a) Device layout and dimensions of a microfluidic device containing herringbone mixers. b) Representative image of captured CEM cells (DiI+, DAPI+) from whole blood; the DAPI+cells (blue only) are nonspecifically captured white blood cells. c) Cancer cell capture efficiency in whole blood at various flow rates using AuNP-aptamer and aptamer alone. d) Calibration plot of cancer cell capture from whole blood and lysed blood with different cell concentrations at 1 μLs, solid lines represent linear fitting. Error bars represent standard deviations (n=3).

FIGS. 6A-6B. Dynamic light scattering (DLS) analysis of a) AuNPs; b) AuNP-sgc8 aptamer conjugates. The hydrodynamic diameter of AuNP increased from 17.4 nm to 61.8 nm after conjugation with aptamers.

FIGS. 7A-7B. Pictures of a) the single flat channel device; b) the parallelized flat channel device with 8 channels connected.

FIG. 8. Adsorption spectrum of AuNPs, (λₘₐₓ=520 nm), using a molar absorptivity of 2.7×10⁸ L mol⁻¹ cm⁻¹, the concentration of the AuNP is ˜13 nM.

FIG. 9. Fluorescence spectrum of fluorescein-labeled aptamers at (a) 10 nM and (b) 1 μM. (c) The fluorescence of AuNP-aptamer conjugates at 10 nM. Around 95 fluorescein-labeled aptamers were conjugated to each AuNP. Thus, the fluorescence signal of each AuNP-aptamer is much higher than individual aptamer, as shown in (a) and (c).

FIGS. 10A-10D. a) Picture of the 3 in.×1 in. microfluidic Geometrically Enhanced Mixing (GEM) chip, consisting of eight parallel channels with single inlet and outlet. b) Micrograph (4× bright field) of the staggered herringbone grooves inside a channel, showing their asymmetry and periodicity, scale bar=200 μm. c) A narrow groove design based on reported herringbone (HB) chip, with 50-μm groove width, purple dots show cells captured inside a channel. d) Cross-sectional view of the wide groove GEM chip, with channel depth of 50-μm and groove depth of 50-μm; the groove pitch is set to be 200 μm, and the groove width is chosen to be 120 μm.

FIGS. 11A-11B. Representative image of a) 1:1 mixture of target L3.6pl cells (red) and control MIA PaCa-2 cells (blue) before sorting; b) L3.6pl cells (red) and MIA PaCa-2 cells (blue) after sorting. Target cells were efficiently captured while most control cells were removed.

FIGS. 12A-12B. a) L3.6pl cell capture efficiency as a function of flow rate; reduced capture occurred at a high flow rate because of a larger shear force and the reduced interaction time between cells and antibody-coated surfaces. b) Capture efficiency of L3.6pl cells and BxPC-3 cells at the optimal flow rate of 1 μL/s, with >90% capture efficiency for both types of cells. Error bars show range (n=3).

FIG. 13. Comparisons of capture efficiency and purity of L3.6pl cells with different groove width: 50-μm (conventional narrow groove HB chip), 80-μm, and 120-μm (wide groove GEM chip). Capture purity is defined as the ratio of the number of target cells captured to the number of total cells captured. Error bars represent range (n=3).

FIGS. 14A-14B. Regression analysis of the number of the L3.6pl cells captured by the microfluidic device versus the number of the cells spiked in 1 mL of a) lysed blood, b) whole blood. The x-axis indicates the number of spiked cells, y is the number of captured cells. Error bars show range (n=3).

FIGS. 15A-15B. a) By high flow rate washing alone, a release efficiency of 34% was obtained; with a combination of trypsinization and high flow rate washing, the release efficiency reached 62% for L3.6pl cells. b) Cell viability before cell capture process (extracted directly from culture) is ˜99%. Cell viability immediately after cell capture in device is ˜89%; after release the viability is ˜86%, without significant difference. The high viability indicates that released cells are suitable for subsequent cell culture. Error bars represent range (n=3).

FIGS. 16A-16C. Phase contrast micrograph (10×) of a) re-cultured BxPC-3 cells; b) re-cultured L3.6pl cells after 9 days of growth. Scale bar=100 μm. c) Flow cytometry test showing that the captured and then recultured cells maintained their binding capability with anti-EpCAM, without any differences compared to intact cells.

FIGS. 17A-17B. Fluorescence microscope images (40×) of CTCs captured from patient blood: a) A representative image of CTCs, with DAPI+, Cytokeratin+and CD45−; b) typical image of white blood cells (WBCs), with DAPI+, CK−, and CD45+. Scale bar=10 μm.

FIGS. 18A-18E. a-c) The number of CTCs per mL of blood from pancreatic cancer patients at different treatment cycles for three patients: a) patient #1; b) patient #2; c) patient #3. d-e) CT scan image of patient #3 at d) the beginning of the treatment (cycle 1); e) the latter stage of treatment (cycle 11); the red arrows indicate regression of the primary pancreatic cancer. Each treatment cycle is 14 days.

FIG. 19. A geometrically enhanced mixing (GEM) chip for high performance pancreatic CTC capture, release, culture, and for monitoring cancer treatment response.

FIG. 20. Flow cytometry test of anti-EpCAM binding with L3.6pl cells. Streptavidin phycoerythrin Cy5 (SA PE-Cy5) was used to label the biotinylated anti-EpCAM.

FIG. 21. Flow cytometry test of anti-EpCAM binding with BxPC-3 cells.

FIG. 22. Flow cytometry test of the binding behaviour between anti-EpCAM with MIA PaCa-2 cells. Data shows that anti-EpCAM does not bind with MIA PaCa-2 cells, indicating that MIA PaCa-2 cells do not express EpCAM.

FIGS. 23A-23B. a) Fluorescence image of the L3.6pl cells after capture and release with PI/AO staining The orange (red merged with green) color indicates nonviable cells (PI and AO staining), while the green color alone indicates viable cells (AO staining alone); b) Flow cytometry test shows that the captured and then released L3.6pl cells maintain their binding capability with anti-EpCAM, without any differences compared to normal L3.6pl cells.

### BRIEF DESCRIPTION OF THE SEQUENCES

SEQ ID NO: 1. DNA sequence of Sgc8 DNA aptamer.

SEQ ID NO: 2. DNA sequence of TD05 DNA aptamer.

SEQ ID NO: 3. DNA sequence of Sgc3b DNA aptamer.

SEQ ID NO: 4. DNA sequence of Sgd5 DNA aptamer.

SEQ ID NO: 5. DNA sequence of KH2B05 DNA aptamer.

SEQ ID NO: 6. DNA sequence of KH1A02 DNA aptamer.

SEQ ID NO: 7. DNA sequence of KH1C12 DNA aptamer.

SEQ ID NO: 8. DNA sequence of TLS1 1 a DNA aptamer.

SEQ ID NO: 9. DNA sequence of PP3 DNA aptamer.

SEQ ID NO: 10. DNA sequence of TV02 DNA aptamer.

SEQ ID NO: 11. DNA sequence of HCH07 DNA aptamer.

SEQ ID NO: 12. DNA sequence of KDED2a-3 DNA aptamer.

SEQ ID NO: 13. DNA sequence of KCHA10 DNA aptamer.

SEQ ID NO: 14. DNA sequence of S11 e DNA aptamer.

SEQ ID NO: 15. DNA sequence of DOV4 DNA aptamer.

SEQ ID NO: 16. DNA sequence of aptTOV1 DNA aptamer.

SEQ ID NO: 17. DNA sequence of KMF2-1a DNA aptamer.

SEQ ID NO: 18. DNA sequence of EJ2 DNA aptamer.

SEQ ID NO: 19. DNA sequence of CSC01 DNA aptamer.

SEQ ID NO: 20. DNA sequence of SYL3C DNA aptamer.

SEQ ID NO: 21. DNA sequence of Anti-EGFR DNA aptamer.

SEQ ID NO: 22. DNA sequence of Anti-PSMA DNA aptamer.

SEQ ID NO: 23. DNA sequence of Sgc8 DNA aptamer (3′ biotinylated).

SEQ ID NO: 24. DNA sequence of Sgc8 DNA aptamer (5′ pegylated and 3′ biotinylated).

SEQ ID NO: 25. DNA sequence of TD05 DNA aptamer (3′ biotinylated).

SEQ ID NO: 26. DNA sequence of TD05 DNA aptamer (5′ pegylated and 3′ biotinylated).

### DETAILED DISCLOSURE OF THE INVENTION

The term “about” is used in this patent application to describe some quantitative aspects of the invention, for example, size. It should be understood that absolute accuracy is not required with respect to those aspects for the invention to operate. When the term “about” is used to describe a quantitative aspect of the invention the relevant aspect may be varied by ±10%.

Various aspects of the disclosed invention provide devices and methods for isolation of a target cell from a population of cells. Various embodiments of the devices of the current invention comprise of one or more microfluidic channels and scaffolding particles conjugated with one or more ligands that bind to the target cells, wherein the scaffolding particle-ligand conjugates are attached on to the surface of the one or more microfluidic channels. The disclosed devices and methods provide high capture efficiency and high capture purity of the target cells. Capture efficiency is defined as the ratio of the number of the target cells captured by a device to the number of the target cells present in the total population of cells passed through the device. Capture purity is defined as the ratio of the number of the target cells captured by a device to the total number of cells captured by the device. Various embodiments provide devices having a capture efficiency of about 80% to about 99%, about 85% to about 95%, or about 90% to about 95%. In certain embodiments, the devices of the current invention provide the capture efficiency of about 90, 91, 92, 93, 94, 95, 96, 97, 98, 99, or 100%.

The devices of the current invention also provide the capture purity of about 80% to about 99%, about 85% to about 95%, or about 90% to about 95%. In certain embodiments, the devices of the current invention provide the capture purity of about 90, 91, 92, 93, 94, 95, 96, 97, 98, 99, or 100%.

The devices of the current invention comprise of one or more microfluidic channels. A microfluidic channel has at least one dimension of less than about 1 mm and can be of any desired shape (e.g., circular, a half-circle, D-shaped, square, rectangular, quadrangular, triangular (V-shaped), etc.). For example, if a microfluidic channel has a quadrangular cross section, the width or the height (or both the width and height) of the microfluidic channel is less than 1 mm. While the length of the microfluidic channel can be of any desired length, certain embodiments provide for channels having a length of about 10 mm to about 200 mm, about 20 mm to about 100 mm or about 30 mm to about 70 mm or about 40 mm to about 60 mm.

In various embodiments, the microfluidic channel may be a quadrangular channel that has the width of about 0.1 mm to about 5 mm, about 0.5 mm to about 4 mm, about 1 mm to about 4 mm, or about 2 mm to about 3 mm. In other embodiments of the invention, the microfluidic channel has a depth of about 10 μm to about 1000 μm, about 20 μm to about 500 μm, about 50 μm to about 200 μm, or about 90 μm to about 150 μm. In a further embodiment, the microfluidic channel of the device of the current invention is a flat microfluidic channel having a length of about 50 mm, width of about 2 mm, and depth of about 100 μm.

In other embodiments, the device comprises one or more microfluidic channel that is a circular channel having a diameter of about 20 μm to about 1000 μm, about 50 μm to about 500μ, about 70 μm to about 200 μm, or about 90 μm to about 150 μm. In certain embodiments, the microfluidic channel has a diameter of about 100 μm.

The device of the current invention can be made from silicon, glass, thermoset polymers (e.g., poly(dimethylsiloxane) (PDMS), polyurethane, epoxy, polyimide,), and thermoplastics (e.g., polycarbonate, acrylic such as poly(methyl methacrylate), polyethylene, polypropylene, polystryrene, Teflon, cyclic olefin polymers, co-polymers, or mixtures thereof).

The devices of the current invention further comprise of scaffolding particles attached to the surface of the one or more microfluidic channels. The scaffolding particles can be nanoparticles. The nanoparticles can be metallic nanoparticles or non-metal nanoparticles. Examples of metallic nanoparticles that can be used in the devices of the current invention, include, but are not limited to, gold, silver, titanium, platinum, iron, molybdenum, manganese, nickel, cobalt, palladium, tin, zinc, lead, copper, aluminum, alloys thereof, and compounds (e.g. oxides) thereof. Examples of non-metal nanoparticles that can be used in the devices of the current invention, include but are not limited to, polymeric nanoparticles (e.g., nanoparticles comprising polypropylene, polystyrene, polyethylene glycol (PEG), polyethylene oxide (PEO), polylactic acid (PLA), polyglycolic acid (PGA), polyhydroxybutanoates (PHB), PEG-PLA (polylactide), PEG-PGA (polyglycolide), poly(glycolic-co-lactic acid), polylactones, poly(dioxanone), poly(caprolactone), polyurethane, polyphosphazenes, polyanhydrides, polycarbonates, polyorthoesters, co-polymers, or mixtures thereof), glass, silica, carbon nanoparticles, silicon nanoparticles, and other inorganic materials. Additional examples of metallic and non-metallic nanoparticles are well known to a person of ordinary skill in the art and are within the purview of the current invention. In one embodiment, gold nanoparticles are used in the devices of the current invention.

The scaffolding particles can be attached to the surface of the microfluidic channels in various ways. In one embodiment, the scaffolding particles are attached to the surface of the microfluidic channels through a spacer which helps the scaffolding particles to float in the lumen of the microfluidic channels. Scaffolding particles floating in the lumen of the microfluidic channels have enhanced interactions with fluids passing through the channels.

In an embodiment, the spacer is a polymer, preferably, a biocompatible polymer. Examples of polymers that can be used to as spacers include, but are not limited to, polyethylene glycol (PEG), oligonucleotides, peptides, polyethylene oxide (PEO), polylactic acid (PLA), polyglycolic acid (PGA), polyhydroxybutanoates (PHB), PEG-PLA (polylactide), PEG-PGA (polyglycolide), poly(glycolic-co-lactic acid), polylactones, poly(dioxanone), poly(caprolactone), polyurethane, polyphosphazenes, polyanhydrides, polycarbonates, and polyorthoesters. In one embodiment, gold nanoparticles are attached to the surface of microfluidic channels by PEG spacers. Additional polymers that can be used to attach scaffolding nanoparticles to the surface of the microfluidic channels are well known to a person of ordinary skill in the art and are within the purview of this invention.

In another embodiment, the spacer contains a cleavable linker. The linker can be cleavable by light (photons), pH, or other physical or chemical means. The linker can also be a specific oligonucleotide sequence that can be cleaved by an enzyme (e.g., deoxyribonuclease for a DNA sequence).

In various aspects of the invention, scaffolding particles are conjugated with one or more ligands. These ligands bind to molecules present on the surface of target cells thereby capturing these cells from the population of cells. Non-limiting examples of ligands that can be conjugated with the scaffolding particles include DNA aptamers, RNA aptamers, XNA (nucleic acid analogs or artificial nucleic acids) aptamers, peptide aptamers, antibodies, receptor binding proteins or ligands (e.g., hormones, steroids, etc.) and small molecule chemicals. Examples of XNA include, but are not limited to, peptide nucleic acid (PNA), Morpholino and locked nucleic acid (LNA), glycol nucleic acid (GNA), and threose nucleic acid (TNA).

Various embodiments of the invention provide scaffolding particles that are conjugated with a plurality of different ligands that bind to different target sites (e.g., receptors) on the surface of the target cells. The ligands attached to the particles can be of a single type (e.g., aptamer only particles, antibody only particles, etc.) or combinations of different types/classes of ligands can be attached to the particles (e.g., antibodies, aptamers, peptide ligands for receptors, hormone receptor ligands and various combinations thereof can be attached to the particles; see further discussion below). “Multivalent scaffolding particles” refers to particles conjugated with a plurality of ligands that bind to multiple target sites on a cell. Thus, multivalent scaffolding particles bind to multiple sites on the target cells can bind the target cells with higher affinity and/or avidity as compared to scaffolding particles conjugated with only a single type of ligand which can, typically, bind only to a single site (e.g., receptor or other structure) on the target cells.

In one embodiment, the scaffolding particles are conjugated with plurality of DNA aptamers. In another embodiment, gold nanoparticles are conjugated with up to 95 different types of DNA aptamers to produce multivalent scaffolding gold nanoparticles wherein each gold nanoparticle can have up to 95 different types of DNA aptamers. Other embodiments provide for up to 1000 (or more than 1000) different types of DNA aptamers to be attached to a scaffolding particle, such as a gold nanoparticle. Various exemplary aptamers that can be attached to the scaffolding particles are provided in Table 2.

In another embodiment, the scaffolding particles are conjugated with plurality of antibodies that bind to different target sites on the target cells. Such embodiments provide the capacity to the microfluidic devices of the current invention to capture various types of target cells in a single device thereby broadening the domain of target cells captured in a single run.

These embodiments are particularly useful in capturing CTCs from body fluids of a subject. Currently available technologies directed to capturing target cells, for example, capturing CTCs are not sensitive enough to detect rare CTCs and are not inclusive enough to detect all CTCs for comprehensive and consistent molecular and functional analysis. Most of these methods can isolate only epithelial tumor cells to the exclusion of EMT cells. In an embodiment of the current invention nanoparticles are conjugated with antibodies against plurality of markers comprising epithelial markers (e.g. EpCAM) and mesenchymal markers (e.g. collagen I). These embodiments can capture CTCs with epithelial markers and CTCs with EMT markers. The ability to successfully capture both epithelial tumor cells (EpCAM+) and EMT tumor cells (EpCAM−) is extremely important in clinic because of well-known capacity of epithelial tumor cells to morph into mesenchymal cells to acquire invasive, migratory and metastatic properties.¹⁹⁻²²

In another embodiment, the scaffolding particles are conjugated a mixture of ligands of different types. For example, the scaffolding particles can be conjugated with a mixture of ligands comprising combinations of DNA aptamers, RNA aptamers, XNA (nucleic acid analogs or artificial nucleic acids) aptamers, peptide aptamers, antibodies, receptor binding proteins or ligands (e.g., hormones, steroids, etc.) and small molecule chemicals, or various sub-combinations of these ligands. For example, nanoparticles can be conjugated with DNA aptamers and antibodies capable of binding to different sites on the target cells.

Various embodiments of the invention provide for the use of scaffolding particles having specificity for a plurality of cells (e.g., cell type A, B, C and D). The scaffolding particles having specificity for each of these cell types can be mixed together and attached to the surface of a microfluidic channel. Alternatively, “zones” having specificity for a specific cell type can be created in a microfluidic channel (e.g., a first zone specific for cell type “A”, a second zone specific for cell type “B”, a third zone specific for cell type “C” and a fourth zone specific for cell type “D” and so on).

The devices of the current invention can further comprise of a micro-mixer or a number of mixers which mix the fluids that pass through the microfluidic channels. The micro-mixer can be a passive micro-mixer or an active micro-mixer. Passive mixers rely on microfeatures created in channels, whereas active mixers use external forces to achieve mixing. Non-limiting examples of micro-mixers that can be used in the devices of the current invention include T- or Y-shaped micro-mixers, parallel lamination micro-mixers, sequential lamination micro-mixers, sequential micro-mixers, focusing enhanced micro-mixers, droplet micro-mixers, pressure field micro-mixers, electrokinetic micro-mixers, dielectrophoretic micro-mixers, electrowetting micro-mixers, magneto-hydrodynamic micro-mixers, ultrasound micro-mixers, asymmetric serpentine micro-mixers, circulation-disturbance micro-mixers, connected-groove micro-mixers, crossing manifold micro-mixers, elecrokinetic instability micro-mixers, electrowetting on dielectrics micro-mixers, magneto hydrodynamic micro-mixers, temperature-induced micro-mixers, planar serpentine micro-mixers, split-and-recombine micro-mixers, slanted-groove micro-mixers, staggered-herringbone micro-mixers, staggered overlapping crisscross micro-mixers, and herringbone groove-based micro-mixers. In an embodiment, the devices of the current invention comprise of a herringbone groove-based micro-mixer. Additional examples of micro-mixer devices are well known to a person of ordinary skill in the art and are within the purview of the current invention.⁵⁸

The devices of the current invention can further comprise of a valve or a number of valves for controlling flow directions, regulating flows, and isolating one region from another in a microfluidic device. The microvalves can be actuated using either passive or active actuation mechanisms, including electric, magnetic, piezoelectric, pneumatic, thermal, and/or phase change.

In various aspects of the invention, the fluid passes through the one or more microfluidic channels at a constant flow rate or a variable flow rate. The flow rate can be about 0.1 to about 50.0 μL/second, about 0.5 to about 10.0 μL/second or about 1.0 to about 2.0 μL/second. In one embodiment, the flow rate is about 1.2 μL/second.

The current invention also provides methods of isolating target cells from a population of cells. The population of cells can be obtained from a cell culture source or from a subject having a disease (e.g., cancer or a disease caused by a pathogen such as a bacterial cell, yeast cell or virus). Isolation of target cells from a population of cells according to the methods of current invention comprises:

a) passing the population of cells through the microfluidic channels of a device of the current invention to facilitate interaction and capture of the target cells by the scaffolding particle-ligand conjugates,

b) washing the microfluidic channels by a washing solution to remove the cells non-specifically bound to the scaffolding particle-ligand conjugates,

c) optionally, passing one or a number of reagents to verify that the captured cells are truly target cells,

d) optionally, enumerating the cells captured,

e) releasing the captured target cells from the scaffolding particle-ligand conjugates, and

f) collecting the released target cells.

In one embodiment, not all of these steps are needed for a certain application. For example, step c is not needed, for example, if target cells are prestained or interacted with dye-labeled molecules.

To isolate target cells from a population of cells, the population of cells from a tissue or body fluids of a subject (an individual) can be processed to prepare a sample containing the population of cells. The subject can be an animal, for example, a mammal such as a human. The population of cells can be separated from other components of the body fluids, for example, by centrifugation or filtration. A population of cells from a solid tissue, for example, a tumor, can also be subjected to the methods of the current invention by homogenizing the solid tissue to prepare a slurry or solution containing the population of cells.

In other embodiments, target cells from the blood from a subject (e.g., a human) are isolated according to the methods of current invention after treatment to lyse the RBCs in the blood without damaging the other cellular components in the blood. Detailed procedures for lysis of RBCs without damaging other components of blood are described elsewhere in this application or are known to those skilled in the art.

In another embodiment, target cells from the body fluids of a subject (e.g., a human) are isolated according to the methods of current invention without any processing or pretreatment except for anti-coagulants contained in the tube used for the blood collection. Whole human blood can be directly introduced into the device. For example, target cells from unprocessed blood obtained from a human can be isolated according to the methods of current invention. Non-limiting examples of body fluids that can be subjected to the methods of current invention include amniotic fluid, aqueous humor, vitreous humor, bile, blood, cerebrospinal fluid, chyle, endolymph, perilymph, female ejaculate, male ejaculate, lymph, mucus (including nasal drainage and phlegm), pericardial fluid, peritoneal fluid, pleural fluid, pus, rheum, saliva, sputum, synovial fluid, vaginal secretion, and blood.

In an aspect of the invention, the methods of isolating the target cells include washing the microfluidic channels with attached scaffolding particle-ligand conjugates with solutions under conditions that allow the captured cells that are bound specifically to the scaffolding particles to remain captured while causing the cells that are bound non-specifically to the scaffolding particles to be washed off The types of solutions or other conditions used to wash off cells that are non-specifically bound to the scaffolding particle-ligand conjugates depend on the specificity and type of molecular interactions between the scaffolding particle-ligand conjugates and the target cells. Such conditions are well known to a person of ordinary skill in the art and are within the purview of the current invention. Non-limiting examples of conditions that cause non-specifically bound cells to be washed off include, but are not limited to, absence/presence and concentration of specific chemicals or biomolecules, pH of the solution, temperature of the solution, shear stress of washing solution, etc.

In one aspect of the invention, the methods of isolating the target cells comprise releasing the target cells captured by the scaffolding particle-ligand conjugates. Releasing the target cells captured by the scaffolding particle-ligand conjugates may comprise treating the captured cells-scaffolding particle complexes under conditions that allow the captured cells bound to the scaffolding particle-ligand conjugates to be released. The types of solutions or other conditions used to release the specifically bound target cells depend on the specificity and type of molecular interactions between the captured cells-scaffolding particle complexes. Such conditions are well known to a person of ordinary skill in the art and are within the purview of the current invention. Non-limiting examples of conditions that cause specifically bound target cells to be released include, but are not limited to, absence/presence and concentration of specific chemicals or biomolecules, pH of the solution, temperature of the solution, shear stress of washing solution, etc.

In an embodiment of the invention, target cells captured by the scaffolding particle-ligand conjugates are released by treatment with agents that interfere with the interactions between the ligands and the target cells thereby separating the target cells from the scaffolding nanoparticle-ligand conjugates. For example, target cells bound to the scaffolding particle-ligand conjugates can be released by treatment with solutions containing high concentration of free ligands thereby interfering with interactions between the target cells and scaffolding particle-ligand conjugates and releasing the target cells. Other embodiments provide for the release of target cells captured by the scaffolding particle-ligand conjugates are released by treatment with agents that interfere with the interaction between the ligands and the scaffolding particles thereby separating the target cells from the scaffolding nanoparticles. For example, target cells captured by gold nanoparticles conjugated with peptide aptamers can be released by treatment with peptidases that cleave the peptide aptamers thereby releasing the captured cells from the scaffolding nanoparticles. In another example, target cells captured by gold nanoparticles conjugated with DNA aptamers can be released by treatment with nucleases that cleave the DNA aptamers from gold nanoparticle-DNA aptamer conjugates thereby releasing the captured cells from the scaffolding nanoparticles. In another embodiment of the invention, the target cells can be released by other physical or chemical means. For example, air or other gas can be used to force the detachment of cells from the scaffold due to the amount of force exerted by the air/liquid interface. In another embodiment of the invention, a cleavable linker is contained in the spacer linking ligands (e.g., aptamers, antibodies, peptides) to the nanoparticles. For example, the linker can be photocleavable and the target cells can be released by using UV light exposure, which have minimal effect on cell viability. A pH cleavable linker can also be used, though it is ideally used under conditions that will not affect cell viability. In the situation where cell viability is not critical (e.g., only genetic analysis is to be performed), a strong acid or base can be used. Alternatively, a peptide linker can be used, and cleaved by a protease. Yet other embodiments provide for the use of an oligonucleotide linker which can be cleaved by an enzyme (e.g., deoxyribonuclease, DNase). In yet another embodiment of the invention, regular biotin can be replaced with cleavable biotin group. In another embodiment of the invention, target cells can be released by DNA hybridization with aptamers. Various combinations of linkers (e.g., photocleavable, pH cleavable, protease cleavable and/or endonuclease cleavable linkers) can be used to attach ligands to a particle.

Materials and Methods

Synthesis and Characterization of Gold Nanoparticle-Aptamer Conjugates

Hydrogen tetrachloroaurate (III) (HAuC1₄), trisodium citrate dihydrate, tris-(2-carboxyethyl)phosphine hydrochloride (TCEP), tris-(hydroxymethyl) aminomethane (Tris), and sodium acetate were obtained from Sigma-Aldrich (St. Louis, Mo.). Acetate buffer (500 mM, pH 5.2) was prepared using a mixture of sodium acetate and acetic acid. Tris acetate buffer (500 mM, pH 8.2) was prepared using Tris and acetic acid.

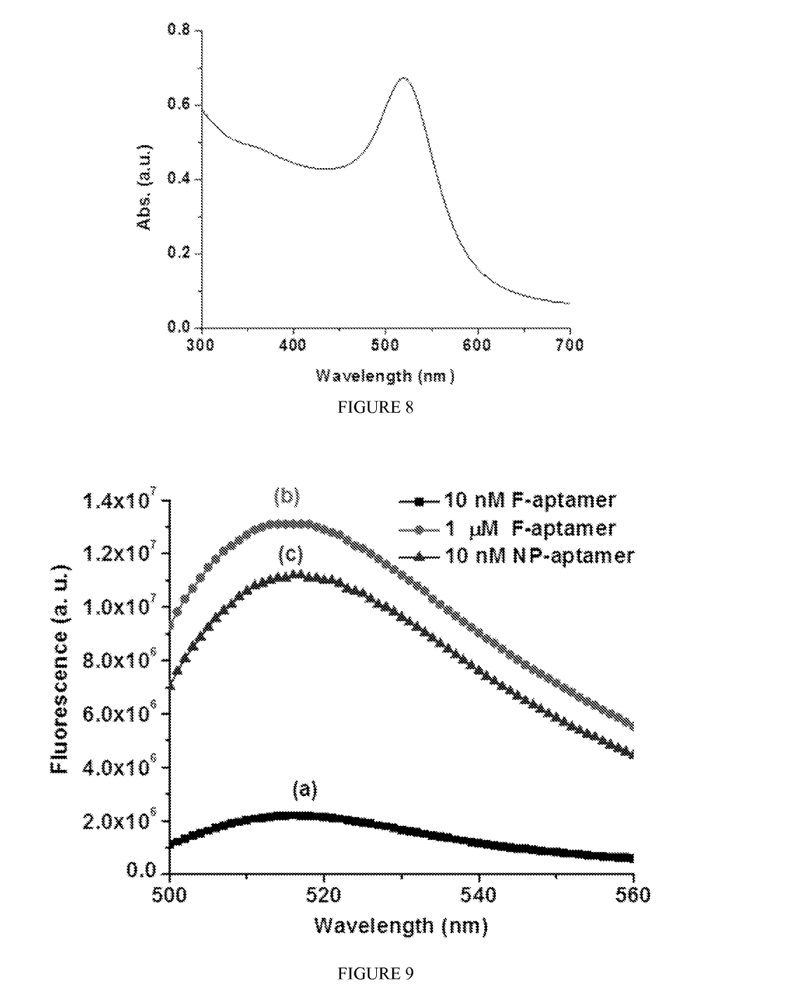

AuNPs were prepared using the protocols reported previously.⁵³ Briefly, 100 mL of 1 mM HAuCl₄ solution was heated till reflux. Then, 10 mL of 38.8 mM sodium citrate was added and reflux was continued for another 20 min. The diameter of such prepared AuNPs was ˜13 nm, measured by transmission electron microscopy (TEM). The concentration of the AuNPs was ˜13 nM, determined by UV-Vis measurement at 520 nm using a Cary Bio-300 UV spectrometer (Varian) (FIG. 8).

DNA aptamers were synthesized. Thiol modified-sgc8 aptamer sequence was: 5′-thiol-PEG-ATC TAA CTG CTG CGC CGC CGG GAA AAT ACT GTA CGG TTA GA-biotin-3′ (SEQ ID NO: 24). The sequences of all aptamers used are listed in Table 1. For flow cytometric analysis, a fluorescein isothiocyanate (FITC) modifier was used to replace the biotin linker. All DNA aptamers were purified using a ProStar HPLC (Varian, Walnut Creek, Calif.) with a C18 column (Econosil, 5U, 250×4.6 mm) from Alltech Associates (Deerfield, Ill.), with triethylammonium acetate-acetonitrile as eluent. DNA concentration was determined by UV-Vis measurement at 260 nm.

Thiol-modified aptamers were conjugated on AuNPs using the reported protocols.^{46,53,54} Aptamers (9 μL, 1 mM) were added with acetate buffer (1 μL, 500 mM) and TCEP (1.5 μL, 10 mM) and incubated for 1 h at room temperature to activate the thiol group. Then the TCEP-treated aptamer was added to 3 mL of as-prepared AuNPs and incubated for 16 h. Finally, Tris acetate buffer (30 μL, 500 mM) and NaCl (300 μL, 1M) were added, and the mixture was incubated for 24 h. Unconjugated aptamers was then removed by centrifugation at 14,000 rpm for 15 min.

TABLE 1
Detailed aptamer sequence information.

| Name | Sequence |
|---|---|
| sgc8 | 5′-ATC TAA CTG CTG CGC CGC CGG GAA AAT ACT |
| | GTA CGG TTA GAT TTT TTT TTT-biotin-3′ |
| | (SEQ ID NO: 23) |
| Thiol- | 5′-thiol-(PEG)₂₄-ATC TAA CTG CTG CGC CGC CGG |
| sgc8 | GAA AAT ACT GTA CGG TTA GA-biotin-3′ |
| | (SEQ ID NO: 24) |
| TD05 | 5′-AAC ACC GTG GAG GAT AGT TCG GTG GCT GTT CAG |
| | GGT CTC CTC CCG GTG TTT TTT TTT T-biotin-3′ |
| | (SEQ ID NO: 25) |
| Thiol- | 5′-thiol-(PEG)₂₄-AAC ACC GTG GAG GAT AGT TCG |
| TD05 | GTG GCT GTT CAG GGT CTC CTC CCG GTG-biotin-3′ |
| | (SEQ ID NO: 26) |

Underlines indicate the full sequence of sgc8
aptamer or TD05 aptamer; for flow
cytometric test, fluorescein isothiocyanate
(FITC) is used instead of biotin linker.

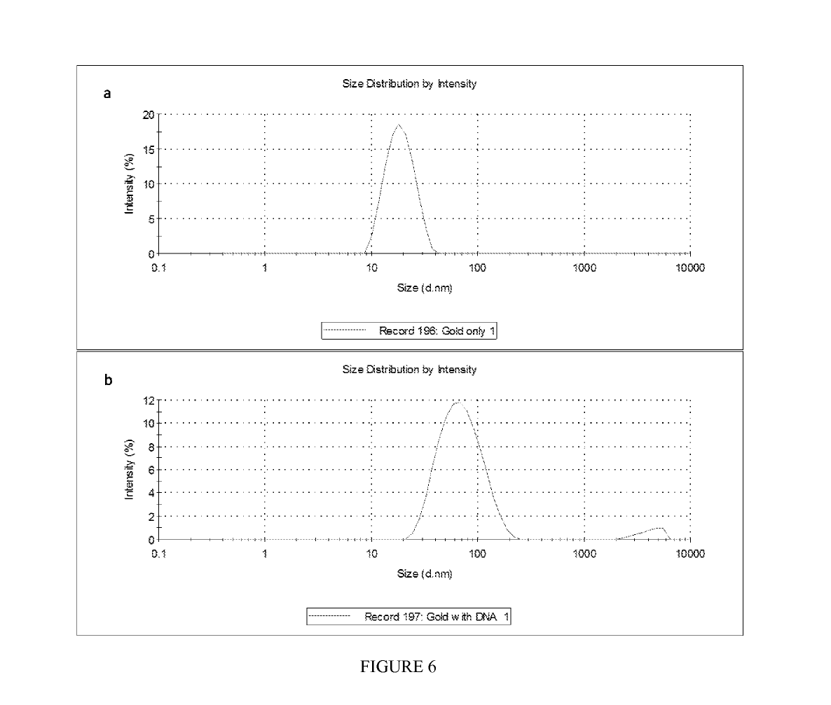

The aptamer concentration in the supernatant was measured, and the final conjugated aptamer concentration in the AuNPs was determined by subtracting the supernatant concentration from the previous aptamer concentration. The final AuNP concentration was 12.7 nM with an aptamer concentration was 1.2 μM, giving an average of approximately 95 aptamers on each AuNP. Dynamic light scattering (DLS) measurement was performed to evaluate the hydrodynamic diameter of the AuNPs before and after conjugation with aptamers using Zetasizer Nano ZS, (Malvern, Worcestershire, United Kingdom) (FIG. 6). Zeta-potential measurements were performed using the same instrument. Fluorescence spectroscopy (FIG. 9) also demonstrated the successful conjugation of aptamer on the AuNP. The fluorescence signal of each AuNP-aptamer conjugate is much higher than that of individual aptamer.

Device Design and Fabrication

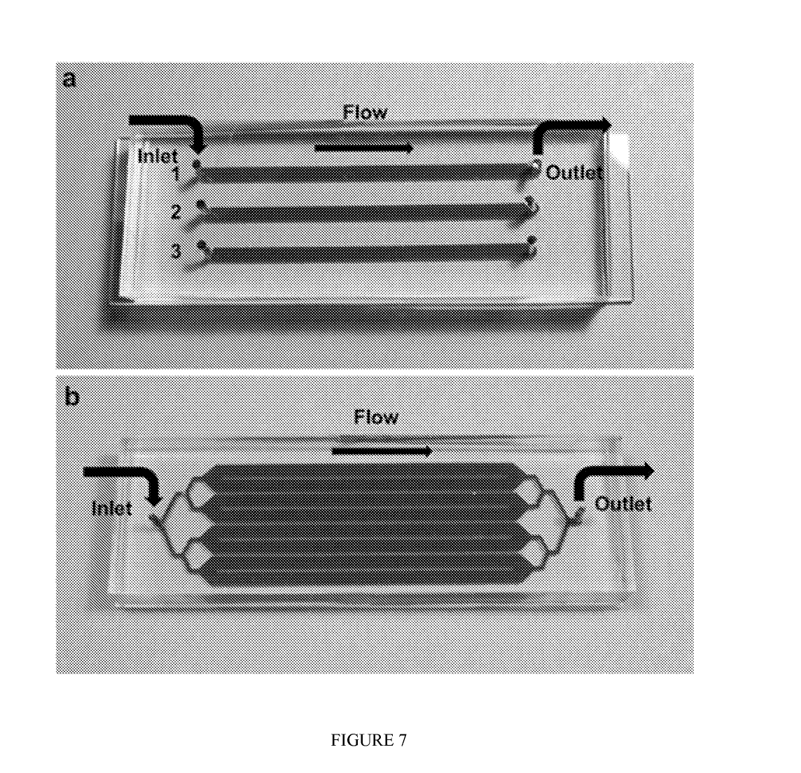

A single flat channel device was initially used for proof-of-concept studies, and then eight flat channels were parallelized to form a high throughput device. As shown in FIG. 7a, the single flat channel device was designed with a length of 50 mm, width of 2 mm, height of 100 μm, and with single inlet and outlet. Three independent devices can be incorporated within one microscope slide size (3 in.×1 in.). To increase the throughput, eight channels were connected through parallelization, and uniform flow was maintained in the eight channels. The size of the high throughput device is also 3 in.×1 in., as shown in FIG. 7b. Both of the two devices were made of polydimethylsiloxane (PDMS), and bonded to a 3 in.×1 in. glass slide.

PDMS devices were fabricated according to the procedures reported by Whitesides' group.⁵⁵ The layout of the device was designed in AutoCAD and then sent to CAD/Art Services, Inc. (Bandon, Oreg.) to produce a high resolution transparency photomask. Silicon wafers (Silicon Inc., Boise, Id.) were first spin-coated with SU-8 2035 photoresist (MicroChem, Newton, Mass.) using a spin coater (Laurell Tech., North Wales, Pa.). Then the pattern on the photomask was transferred to the silicon substrate via UV exposure. After development, a silicon master patterned with the complementary structures was obtained. PDMS devices were fabricated by casting a liquid PDMS precursor against the master using Sylgard 184 reagents (Dow Corning, Midland, Mich.) according to the instructions of the manufacturer. To prevent the cured PDMS from sticking to the silicon master, TFOCS (Tridecafluoro-1,1,2,2-tetrahydrooctyl-1-trichlorosilane) (Sigma-Aldrich, St. Louis, Mo.) was vacuum vaporized to the surface of the master. The channel height, which was controlled by the spin speed of the SU-8, was measured using a Dektak 150 profilometer. The PDMS substrate was then sealed with a glass microscope slide, and inlet and outlet wells were created at the channel ends by punching holes in the PDMS sheet.

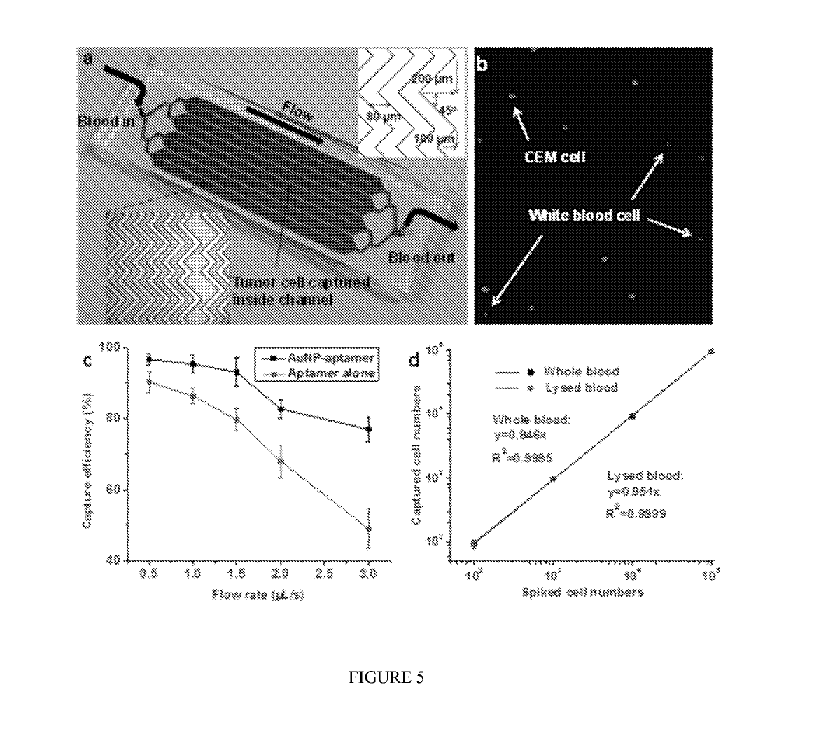

A herringbone mixer-device is shown in FIG. 5a. The mixer device was fabricated as described above, but using a two-layer SU-8 fabrication technique, with two coating and exposure steps and a single developing step.⁵⁷ The silicon mold has a first layer as the main channel and the second layer containing herringbone ridges, which become grooves after transfer to the PDMS substrate.

Flow Cytometric Analysis

Flow cytometry was used to evaluate the targeting capabilities of AuNP-aptamer conjugates toward specific cells. Fluorescence measurements were made with a FAC Scan cytometer (BD Immunocytometry Systems, San Jose, Calif.). Briefly, 200,000 cells were incubated with FITC-labeled free aptamer or AuNP-aptamer conjugates in 200 μL of PBS (containing 0.1% BSA) for 30 min on ice. After incubation, the cells were washed three times by centrifugation with 200 μL PBS, and 10,000 counts were measured in the flow cytometer to determine the fluorescence. Varying concentrations of free sgc8 and AuNP-sgc8 aptamers were used to determine their binding affinities. The fluorescein-labeled random DNA library was used as a negative control to determine nonspecific binding. All of the experiments for the binding assay were repeated three times. The mean fluorescence intensity of target cells labeled by aptamers was used to calculate for specific binding by subtracting the mean fluorescence intensity of nonspecific binding from random library. The equilibrium dissociation constants (K_{d}) of the aptamer-cell interaction were obtained by fitting the dependence of fluorescence intensity of specific binding on the concentration of the aptamers to the equation Y=BₘₐₓX/(K_{d}+X) using SigmaPlot (Jandel, San Rafael, Calif.), where Y is the fluorescence intensity and X is the concentration of aptamers.

Microfluidic Device Fabrication

The microfluidic geometrically enhanced mixing chip (GEM chip) consists of a polydimethylsiloxane (PDMS) structure bonded to a 3″×1″ glass microscope slide. The PDMS structure was fabricated using two-layer soft lithography, according to literature. The two-layer SU-8 structure (a main channel layer and a herringbone mixer layer) was fabricated via two spin-coating and exposure steps and a single developing step. The device layout was designed in AutoCAD and then sent to CAD/Art Services, Inc. (Brandon, Oreg.) to produce a high resolution transparency photomask. Silicon wafers were first spin-coated with 50-μm thick SU-8 2035 photoresist (MicroChem, Newton, Mass.) as the main channel layer. After soft baking, UV light exposure, and post exposure baking, another layer of SU-8 was added to form the herringbone mixer layer. With precise alignment between the main channel and the mixer, a second exposure was performed to create the herringbone mixer pattern. After development, a silicon master patterned with the complementary structures was obtained. PDMS structures were fabricated by casting a liquid PDMS precursor against the master using Sylgard 184 reagents (Dow Corning, Midland, Mich.), according to the manufacturer's instructions. Inlet and outlet wells were created at the channel ends by punching holes in the PDMS sheet. The channel depth, which was controlled by the spin speed of the SU-8, was measured using a Dektak 150 profilometer.

Cell Culture

T-cell human acute lymphoblastic leukemia cells (CCRF-CEM cells, CCL-119) and B-cell human Burkitt's lymphoma cells (Ramos cells, CRL-1596) were purchased from American Type Culture Collection (ATCC). CEM and Ramos cells were cultured in RPMI medium 1640 (ATCC) supplemented with 10% fetal bovine serum (FBS; heat-inactivated; GIBCO) and 100 units/mL penicillin-streptomycin (Cellgro, Manassas, Va.). Both cultures were incubated at 37° C. under 5% CO₂ atmosphere.

BxPC-3 cells (CRL-1687, human pancreatic adenocarcinoma) and MIA PaCa-2 cells (CRL-1420, human pancreatic carcinoma) were purchased from American Type Culture Collection (ATCC). Cells were cultured in DMEM medium (ATCC) supplemented with 10% fetal bovine serum (FBS; heat-inactivated; GIBCO) and 100 units/mL penicillin-streptomycin (PS, Cellgro, Manassas, Va.) and incubated at 37° C. under 5% CO₂ atmosphere. Cells were grown as adherent monolayers in 60 mm×15 mm culture dishes to 90% confluence, subsequently detached with 0.05% Trypsin-0.53 mM EDTA (0.05%, Cellgro) and re-seeded at a lower concentration.

Reagents and Buffers

Biotinylated anti-EpCAM (Anti-Human CD326, eBioscience, San Diego, Calif.) immobilized on device surface was used as the CTC capture agent. Anti-cytokeratin FITC (CAM 5.2, conjugated with fluorescein isothiocyanate, BD Biosciences, San Jose, Calif.) and anti-CD45 PE (conjugated with phycoerythrin, BD Biosciences) were used to label CTCs and white blood cells, respectively. DAPI (4′,6-diamidino-2-phenylindole, Invitrogen, Carlsbad, Calif.), which stains DNA in cell nuclei, was used to label all nucleated cells bound to the device (i.e., white blood cells and CTCs). Dulbecco's phosphate buffered saline with calcium and magnesium (PBS, Fisher Scientific, Hampton, N.H.) was used to wash cells. A buffer containing 10 mg/mL (1%) bovine serum albumin (BSA, Fisher Scientific) and 0.05% Tween-20 (Fisher Scientific) in PBS was used for rinsing the unbound molecules from the channel surface, and resuspending cells for cell capture. BSA and Tween-20 in PBS was used to fully passivate the surfaces to reduce nonspecific adsorption of cells in the channels.

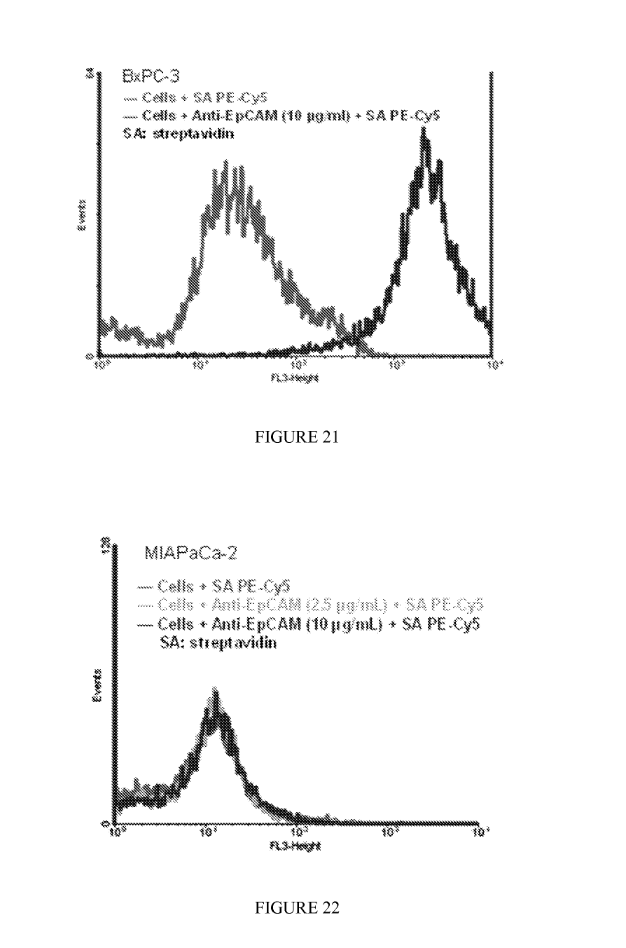

Flow cytometry analysis was used to test the binding capabilities of anti-EpCAM to pancreatic cancer cell lines. Fluorescence measurements were performed with a FACScan cytometer (BD Immunocytometry Systems, San Jose, Calif.). Briefly, 200,000 cells were incubated with 10 μg/mL biotinylated anti-EpCAM in 200 μL of PBS (containing 0.1% BSA) for 20 min on ice. After incubation, the cells were washed three times with PBS. Then streptavidin phycoerythrin (SA PE)-Cy5 (Invitrogen) was added and incubated for another 20 min. After washing, 10,000 counts were measured in the flow cytometer to determine the fluorescence. The cells incubated with SA PE-Cy5 alone were used as a negative control to determine nonspecific binding. FIGS. 20 and 21 show the strong binding of the anti-EpCAM antibody with L3.6pl cells and BxPC-3 cells, respectively. FIG. 22 shows no binding between anti-EpCAM and MIA PaCa-2 cells, indicating that MIA PaCa-2 cells can be used as a negative control.

Capture of Spiked Tumor Cells in Microfluidic Devices

Immediately before experiments, cells were detached from the culture dish and then rinsed with PBS and resuspended at 10⁶ cells/mL. By following the manufacturer's instructions, the target cells and control cells were stained with Vybrant DiI (red) and Vybrant DiD (blue) cell-labeling solutions (Invitrogen), then rinsed with PBS, and resuspended at 10⁶ cells/mL in the PBS containing BSA and Tween-20. Labeled cells were stored on ice and further diluted or spiked into blood to the desired concentrations before experiments.

Anti-coagulant-containing human whole blood from healthy participants was purchased from Innovative Research (Novi, Mich.), and used for all “spike-in” experiments. For some experiments, CTC capture from whole blood samples was preceded by red blood cell lysis performed as previously described. Briefly, lysed blood was obtained by treating whole blood with red blood cell (RBC) lysing buffer, prepared by adding 155 mM (8.3 g/L) ammonium chloride in 0.01 M Tris-HCl buffer, with pH=7.5. Different concentrations of cancer cell lines were then spiked in whole blood or lysed blood.

To initiate cell capture experiments, one channel volume (˜100 μL) of 1 mg/mL avidin (Invitrogen) in PBS was first introduced into the device, followed by incubation for 15 min and then three rinses with PBS. Then, one channel volume of biotinylated anti-EpCAM (20 μg/mL), sgc8 aptamer or AuNP-sgc8 aptamer was introduced into the device and incubated for 15 min, followed by three rinses with the PBS containing BSA and Tween-20. Finally, 1 mL of cell mixture or blood sample was pumped into the device at a flow rate of 1 μL/s or 1.2 μL/s (or other flow rates specified in the text). At the end of the experiment, the microchannel was washed three times with PBS, followed by acquiring fluorescent images for the determination of the number of cells captured.

To test the purity of captured cells from lysed blood or whole blood, DAPI (Invitrogen) was introduced into the device to label the nonspecifically captured white blood cells. By following the manufacturer's instructions, cells were incubated with 300 nM DAPI for 10 min, followed by rinsing with PBS.

Instrument Setup

The cell suspension or blood sample was introduced into the device by pumping using a syringe pump (KD Legato 111, KD Scientific, Holliston, Mass.) with a BD syringe connected to the inlet of the device via polymer tubing and a female luer-to-barb adapter (IDEX Health & Science, Oak Harbor, Wash.). To avoid cell settling, a tiny magnetic stirring bar was placed inside the 1 mL syringe, with a stir plate beneath the syringe. The magnetic stirring bar kept cells in suspension while the cell mixture or blood was being pumped through the device. An Olympus IX71 fluorescence microscope (Olympus America, Melville, N.Y.) with an automated ProScan stage (Prior Scientific, Rockland, Mass.) was used to image and count the captured cells on the device.

To determine cell numbers, a set of three images corresponding to the red fluorescent cells, blue fluorescent cells, and transmission images was acquired at different positions in each channel. Images were then imported into ImageJ (NIH), and cell counts were obtained using the Analyze Particles function after setting an appropriate threshold. Cell numbers were further verified by comparing fluorescent images with transmission images; only those with appropriate cell morphology in the transmission images were counted.

Cell Release and Re-Culture

Cell release was achieved by trypsin and high flow rate washing. After cell captured inside the channel, proteolytic enzyme trypsin (0.25%) was introduced into the device and incubated for 5 min at 37° C. Then, cell culture medium was pumped into the device at a flow rate of 5 μL/s to dislodge the bound cells. The release flow rate was much higher than the cell capturing flow rate of 1 μL/s. Released cells were collected in a new cell culture dish (60 mm×15 mm size), with a total volume of 4 mL culture medium. Then the cells were put into the incubator for propagation in culture.

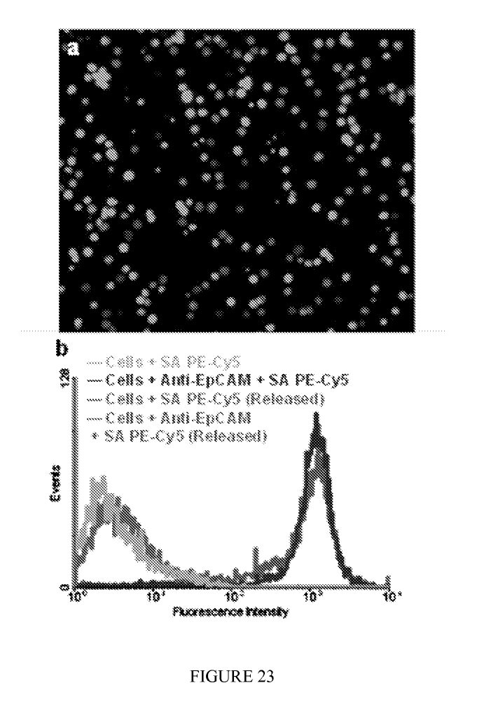

To test the viability of cells captured by the device, propidium iodide (PI) and acridine orange (AO) staining (Invitrogen) assays were performed. PI is a membrane-impermeant stain that labels only dead cells with red fluorescence. AO is a membrane-permeable dye that binds to nucleic acids of all cells and induces green fluorescence. By following the manufacturer's instructions, PI/AO working solution was prepared to contain 2 μM PI and 2 μM AO in PBS. After incubating the working solution with cells for 10 min, fluorescent images were taken to evaluate the viability of the captured cells (FIG. 23a).

Patient Blood Specimen Collection and Processing

Blood samples of patients with metastatic pancreatic cancer and from normal healthy participants were obtained. Specimens were collected into BD Vacutainer tubes containing anti-coagulant sodium heparin and were processed within 6 hours after being drawn. CTC capture was performed by the same protocols as described above. Unlike the pre-stained tumor cells spiked in blood, CTCs from patients' blood were not labeled. Three-color immunocytochemistry (DAPI, FITC anti-cytokeratin, PE anti-CD45) was conducted to identify CTCs from nonspecifically captured blood cells. Cell staining began with cell fixation and permeabilization by incubation for 20 min with 4% paraformaldehyde and 0.2% Triton X-100, respectively. Then, a mixture of 10 μg/mL PE anti-CD45, 10 μg/mL FITC anti-cytokeratin and 500 nM DAPI were introduced into the device and incubated for 20 min. After washing, the microfluidic device was examined under the fluoresce microscope. Only cells that were DAPI positive, CD45 negative, cytokeratin positive, with the appropriate size and morphology were counted as CTCs (DAPI+, CD45−, cytokeratin+). Cell debris, red blood cells (DAPI-) and white blood cells (DAPI+, CD45+, cytokeratin−) and “double positive” cells (both CD45+ and cytokeratin+, with DAPI+) were excluded from counting. CTC capture purity was defined as the ratio of the number of CTCs captured to the total number of nucleated cells (DAPI+) bound to the device. For another sets of experiments, we released the specifically captured CTCs along with nonspecifically captured leukocytes into culture dish (instead of staining and counting). And fresh medium was added once a week (with leukocytes washed away). We observed a few cells (probably CTCs) adhered to the culture dish after 1 week of culture. However, these adhered cells did not proliferate, even after 4 months of culturing (unlike the spiked tumor cells which grew into clusters within 2 weeks).

All patents, patent applications, provisional applications, and publications referred to or cited herein are incorporated by reference in their entirety, including all figures and tables, to the extent they are not inconsistent with the explicit teachings of this specification.

Following are examples which illustrate procedures for practicing the invention. These examples should not be construed as limiting. All percentages are by weight or relative numbers, and all solvent mixture proportions are by volume unless otherwise noted.

### EXAMPLE 1

### Synthesis and Characterization of AuNP-Aptamer Conjugates

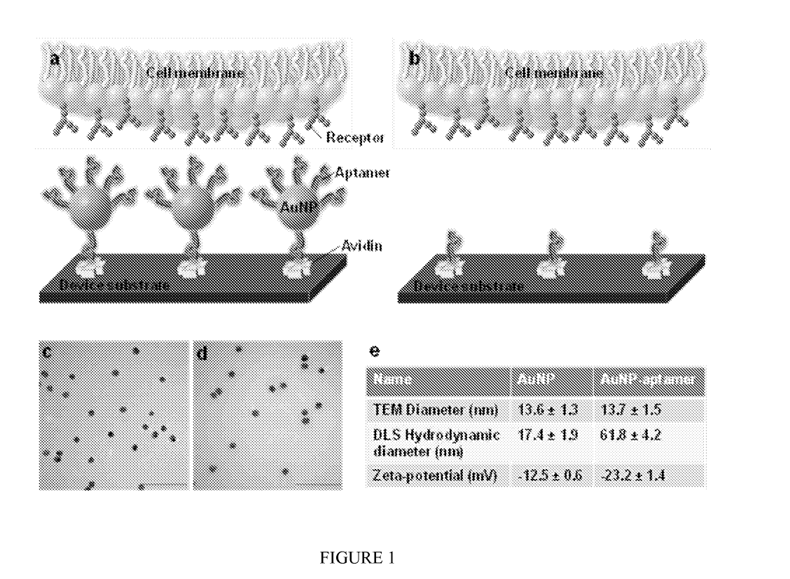

AuNPs were prepared following the methods described above. FIG. 1c shows the transmission electron microscopy (TEM) image of the AuNPs, with an average diameter of 13.6 nm. The as-prepared AuNPs were then functionalized with thiol-modified DNA aptamers, and the TEM image is shown in FIG. 1d, with average size of 13.7 nm. A 24-unit polyethylene glycol (PEG) spacer between AuNP surface and aptamers was added to minimize the steric effects of the particle surface on aptamers and to increase the loading of DNA on AuNPs.⁴⁶ FIGS. 1c and 1d show that the properties of AuNPs remained unchanged after conjugation with aptamers, without any aggregation. Dynamic light scattering (DLS) measurements showed that the hydrodynamic diameter of AuNPs was 17.4 nm. After conjugation with aptamers, the hydrodynamic diameter increased to 61.8 nm, demonstrating the successful conjugation of aptamers onto AuNPs (FIG. 6). Zeta-potential measurements indicated that the AuNPs had a zeta-potential of −12.5 mV. After modification with aptamers, the zeta-potential became −23.2 mV, which is attributed to the negative charges carried by DNA aptamers. The comparison of properties between AuNPs and AuNP-aptamers is made in FIG. 1e.

### EXAMPLE 2

### Flow Cytometric Analysis Demonstrating Multivalent Binding

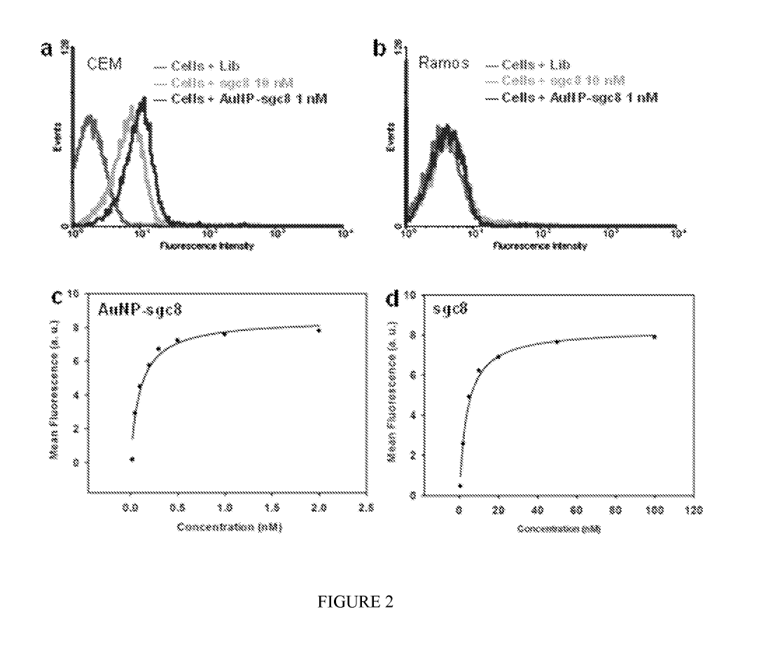

To investigate the AuNP-aptamer mediated multivalent binding, binding behaviors of AuNP-sgc8 aptamer conjugates (AuNP-sgc8) and free sgc8 aptamer (sgc8) using flow cytometry was measured. Sgc8 is an aptamer that has specific binding with CEM cells (human acute lymphoblastic leukemia), with a nanomolar (nM) dissociation constant (K_{d}).³⁶ Ramos cells (human Burkitt's lymphoma) that do not bind with sgc8 aptamer were used as control cells. FIG. 2a shows a noticeable increase in fluorescence signal for both AuNP-sgc8 and free sgc8 aptamer compared to the random DNA library (Lib), proving that both have strong binding with their target cells. Besides, AuNP-sgc8 produces a higher fluorescence signal than free sgc8, even with 10 times lower concentration, showing that AuNP-aptamer increased both the signal and binding strengths of these aptamers for cancer cell recognition. As shown in FIG. 2b, neither free sgc8 nor AuNP-sgc8 shows a signal when incubated with control Ramos cells, demonstrating the specificity of both free aptamers and AuNP-aptamers. Furthermore, the binding affinity of sgc8 and AuNP-sgc8 to CEM cells was measured quantitatively by studying their binding with varying concentrations of sgc8 and AuNP-sgc8 aptamers. As demonstrated in FIGS. 2c & 2d, AuNP-sgc8 shows a 39-times higher binding affinity (K_{d}=0.10±0.02 nM) than that of free sgc8 (K_{d}=3.9±0.5 nM). The lower dissociation constant of AuNP-sgc8 suggests a multivalent-mediated enhancement in binding affinity when multiple aptamers on the AuNP surface bind to multiple receptors on the cell membrane.

### EXAMPLE 3

### Enhanced Cancer Cell Capture in a Flat Channel Microfluidic Device

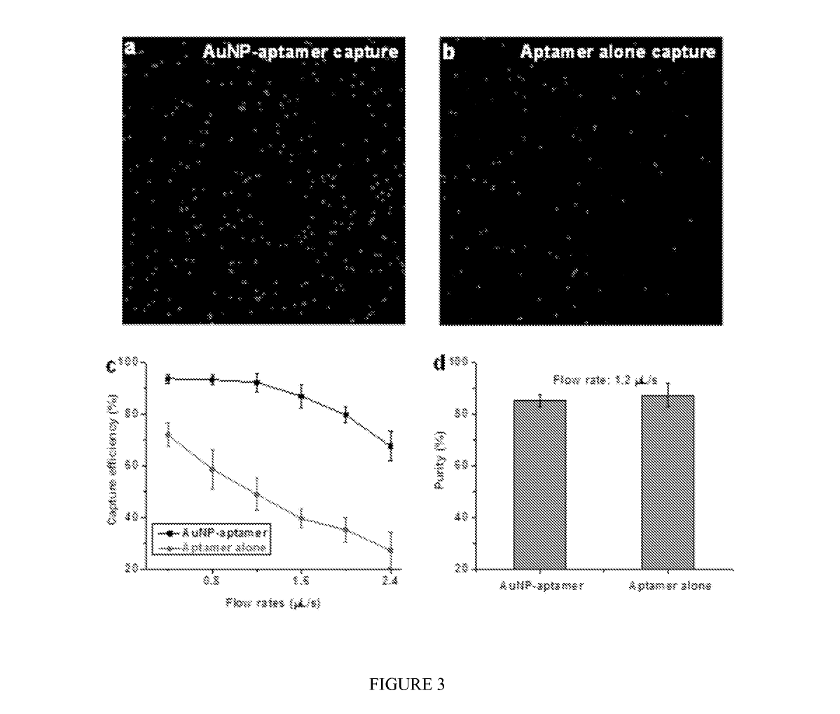

To study the cancer cell capture using AuNP-aptamer, we first developed a microfluidic laminar flow device with flat channels (FIG. 7b), which allowed us to directly compare the capture performance between AuNP-aptamer and aptamer alone. After coating surfaces with AuNP-sgc8 aptamer, a cell mixture containing 10⁵ target CEM cells and 10⁶ control Ramos cells (1:10 ratio) in 1 mL of phosphate buffered saline (PBS) was introduced into the channel. CEM and Ramos cells were pre-stained with Vybrant DiI (red) and DiD (blue), respectively. FIG. 3a shows a representative image of cells captured using AuNP-aptamer, a high percentage of target CEM cells (red) were captured, while most control Ramos cells (blue) were washed away. In another set of experiments with the same conditions, sgc8 alone was used instead of AuNP-sgc8. FIG. 3b shows a typical image of cells captured after washing using aptamer alone (without the nanoparticle conjugation). The results in FIGS. 3a & 3b clearly indicate that many more target CEM cells were captured using AuNP-aptamer than with aptamer alone, demonstrating that enhanced cell capture was achieved via the multivalent binding enabled by the AuNP conjugation. The capture efficiency using AuNP-aptamer and aptamer alone was also studied at different flow rate conditions (with different shear stresses). AuNP-aptamer exhibited more enhancement in the capture efficiency at higher flow rates, as shown in FIG. 3c. At a flow rate of 1.2 μL/s, AuNP-aptamer maintained a capture efficiency of (92±4)%; while aptamer alone yielded a capture efficiency of only (49±6)%. The multivalent binding enables significant increase in capture efficiency for the target cells. The capture purity is not affected by the AuNP conjugation. As shown in FIG. 3d, similar purity was obtained for AuNP-aptamer and aptamer alone.

In addition to the DNA nanosphere-mediated multivalent binding, the enhanced cell capture also accrues from the nanosphere-modified surface, which allows enhanced local topographic interactions between the aptamer-coated nanoparticle and nanoscale components on the cell surface.⁴⁴ Furthermore, the enhanced binding strength afforded by the multivalency effect lowers the detachment ratio of immobilized cells, thus increasing the capture efficiency compared to aptamer alone. This AuNP-aptamer significantly increases the cell capture at high flow rate with high shear stress. Higher shear stress leads to better purity, since non-target cells can be easily washed away.

The devices of the current invention were also used to capture Ramos cells using AuNP-TD05 aptamer conjugates. TD05 is an aptamer with specific binding to Ramos cells.⁴⁷ A capture efficiency of 90% was obtained with AuNP-TD05, while TD05 aptamer alone yielded only 41% capture, showing significant enhancement in capture efficiency as a result of using DNA nanosphere.

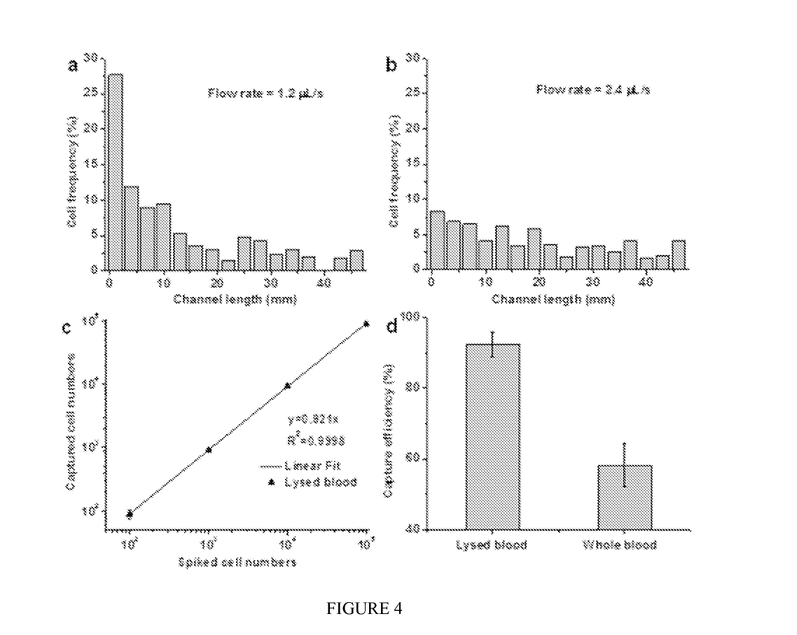

The reduced capture efficiency at higher flow rates (shown in FIG. 3c) can be due to the increased flow-induced shear stress and the decreased interaction time between cells and aptamers on surfaces. The distribution of captured cells at different locations of the 50 mm long microchannel with different flow rates was also characterized. As shown in FIG. 4a, at flow rate of 1.2 μL/s (with a shear stress of 0.4 dyn/cm²), 65% of the cells were captured in the first 25% of the channel coated with AuNP-aptamer. With an increased flow rate of 2.4 μL/s (FIG. 4b), the cells captured were distributed along the channel because cells needed longer diffusion length to have an opportunity to interact with aptamers on the surfaces, and the attached cells experienced proportionally increased shear stresses. With the AuNP-conjugation, the PEG spacer extends the aptamer strands into the 3D space of flow, increasing the accessibility and frequency of interactions between aptamers and cells to permit more efficient cell capture under higher flow rates.

Isolation of CEM cells from lysed blood (blood with red blood cells lysed) at concentrations ranging from 10⁵ to 100 cells/mL was assessed to explore the clinical utility of the devices of the current invention. As shown in FIG. 4c, as few as 100 cells were efficiently isolated from 1 mL of lysed blood within 14 min. However, when we tried to capture cancer cells from unprocessed whole blood directly, the capture was significantly lower (even at a low flow rate), as shown in FIG. 4d. The relatively low capture was primarily due to the reduced interaction chances between target cells and AuNP-aptamer, which can be caused by abundant red blood cell blockage.

### EXAMPLE 4

### Efficient Isolation of Cancer Cells from Whole Blood Using DNA Nanospheres in Micromixer Devices

Although the laminar flow flat channel device achieved high efficiency when capturing cells in PBS and lysed blood, it showed a low capture efficiency (<60%) when capturing cells from whole blood. To provide more efficient capture of CTCs from whole blood, we integrated the AuNP-aptamer system into a herringbone groove-based micro-mixer device (FIG. 5a). The staggered herringbone mixer generates micro-vortex and chaotic mixing inside the microchannel, which significantly enhances the cell-surface interactions, leading to higher capture efficiency.^{12, 48} Isolation of 10⁴ CEM cells (pre-stained by DiI, red) spiked in 1 mL of whole blood was evaluated at a flow rate of 1 μL/s. After cell capture and rinsing, 4,6-diamidino-2-phenylindole (DAPI) was introduced into the device to test the purity of the target cells. DAPI stained all the cancer cells and leukocytes with blue color and verified that captured cells retain intact nuclei. As shown in FIG. 5b, cells positive to both DAPI and DiI were target CEM cells (blue merged with red), while cells positive to DAPI only were white blood cells (blue only). A purity of 70% was obtained when capturing CEM cells from whole blood, with a capture efficiency of 91%. This capture purity from whole blood is much higher than those reported in literature (˜50% & 14%).^{11, 12} Further, the capture efficiency was tested over a wide range of flow rates from 0.5 μL/s to 3 μL/s. Control experiments using identical device and conditions with aptamer alone (no AuNP-conjugation) were then conducted. Much higher capture efficiencies were obtained using AuNP-aptamer, especially at high flow rates (FIG. 5c). The combined effect of multivalent binding from AuNP-aptamer with the passive mixing provided by the herringbone structure enabled high capture efficiency from whole blood (93%) at high flow rate (1.5 μL/s). To test the limit of detection for the cell capture system, cell spike numbers from 100,000 to 100 were explored, and >90% capture efficiency were obtained for all cases. Regardless of whether the red blood cells are intact or lysed, high capture efficiency is always obtained by the integration of AuNP-aptamer with a herringbone mixer (FIG. 5d). With the flow rate of 1.5 μL/s, 1 mL of blood sample can be processed in 11 minutes, which gives sufficient throughput for clinical applications.

### EXAMPLE 5

### Multivalent DNA Nanospheres for Enhanced Capture of CTC from Peripheral Blood

CTCs from peripheral blood or cancer cells from bone marrow have significant applications in cancer diagnosis, therapy monitoring and drug development. CTCs are cancer cells shed from primary tumors; they circulate in the bloodstream, leading to metastasis. The extraordinary rarity of CTCs in the bloodstream makes their isolation a significant technological challenge. This technological challenge can be overcome by combining multivalent DNA aptamer nanospheres with microfluidic devices for efficient isolation of cancer cells from blood. Gold nanoparticles (AuNPs) were used as scaffolds for assembling a number of aptamers to produce multivalent nanoparticles for high-efficiency cell capture. Up to 95 aptamers were attached onto each AuNP, resulting in enhanced molecular recognition capability. An increase of 39-fold in binding affinity was confirmed by flow cytometry for AuNP-aptamer conjugates (AuNP-aptamer) when compared with aptamer alone. With a laminar flow flat channel microfluidic device, the capture efficiency of human acute leukemia cells from a cell mixture in buffer increased from 49% using aptamer alone to 92% using AuNP-aptamer. AuNP-aptamer in a microfluidic device of the current invention can also be used with herringbone mixing microstructures for isolation of leukemia cells in whole blood. The cell capture efficiency was also significantly increased with the AuNP-aptamer over aptamer alone, especially at high flow rates. Thus, the devices of the current invention combining DNA nanostructures with microfluidics has a great potential for sensitive isolation of CTCs, and is a promising tool for cancer diagnosis and prognosis.

The scheme of the AuNP-aptamer mediated multivalent binding for cell capture is shown in FIG. 1. The microfluidic device surface was first coated with avidin by physical adsorption. Then, biotinylated aptamer-conjugated AuNPs were immobilized onto the channel through biotin-avidin interaction. When a sample containing target cancer cells pass through the channel, cells are captured via the specific interaction between the aptamers and the target cell receptors. Since each AuNP is conjugated with ˜95 aptamers, the AuNP-aptamer can bind to cell surface markers in a cooperative manner, leading to multivalent effects and resulting in enhanced cell capture efficiency. In addition to the multivalent binding, the AuNP-aptamer modified surface allows enhanced local topographic interactions between the AuNP-aptamer and nanoscale receptors on the cell surface,^{21, 44-45} contributing to the increased cell capture.

This non-limiting example demonstrates the use of gold nanoparticles as an efficient multivalent vehicle for molecular assembly of aptamers for target cancer cell capture in microfluidic devices. Up to 95 aptamers were attached onto each AuNP, resulting in enhanced aptamer molecular recognition capability. Flow cytometry results demonstrated the multivalent binding effect using AuNP-aptamer conjugates and the capture efficiency for target cancer cells was significantly increased using the AuNP-aptamer conjugates because of the cooperative, multiple ligand-receptor interactions.

With the AuNP-aptamer surface immobilization, a flat channel microfluidic device was able to capture 100 cancer cells from 1 mL of lysed blood with ˜90% capture efficiency within 14 min (4.3 mL blood/h). Using the integration of the AuNP-aptamer with a herringbone mixer design, efficient capture of rare cancer cells from whole blood was achieved, with a throughput of processing 1 mL of blood in 11 min. The high efficiency, throughput, and purity makes the system suitable for clinical isolation of CTCs from patient blood.

An advantage of the AuNP-aptamer based system compared with antibody-based devices is that the DNA aptamer can be cleaved by nucleases, leading to noninvasive release of captured cells,⁴⁹ which will be useful for subsequent CTC culture and cellular analysis. The use of leukemia cell-targeting aptamers allows the platform to be suitable for minimal residual disease (MRD) detection. MRD is the small amount of leukemia cells remaining in patient blood during or after treatment when the patient is at remission, which is the major cause for cancer relapse.⁵⁰⁻⁵¹ Thus, the current invention provides efficient isolation of rare cells suitable for sensitive detection of MRD, which is promising for monitoring treatment response and predicting cancer relapse.

Spherical DNA nanostructures have been well developed and widely used for cancer cell detection; however, the current invention provides the first use of aptamer nanospheres for enhancing cancer cell capture. The results shown herein demonstrate that the combination of nanotechnology with a microfluidic device⁵² has a great potential for sensitive isolation of CTCs from patient blood, and is promising for cancer diagnosis and monitoring treatment response.

TABLE 2

| Aptamer | Sequence |
|---|---|
| Sgc8 | 5′-ATC TAA CTG CTG CGC CGC CGG GAA AAT ACT GTA CGG |
| | TTA GA-3′ |
| | (SEQ ID NO: 1) |
| TD05 | 5′-AAC ACC GTG GAG GAT AGT TCG GTG GCT GTT CAG GGT CTC |
| | CTC CCG GTG-3′ |
| | (SEQ ID NO: 2) |
| sgc3b | 5′-ACT TAT TCA ATT CCT GTG GGA AGG CTA TAG AGG GGC CAG TCT |
| | ATG AAT AAG-3′ |
| | (SEQ ID NO: 3) |
| Sgd5 | 5′-ATA CCA GCT TAT TCA ATT ATC GTG GGT CAC AGC AGC GGT TGT |
| | GAG GAA GAA AGG CGG ATA ACA GAT AAT AAG |
| | ATAGTAAGTGCAATCT-3′ |
| | (SEQ ID NO: 4) |
| KH2B05 | 5′-ATC CAG AGT GAC GCA GCA CAC ACA ACC TGC TCAT AAA CTT |
| | TAC TCT GCT CGA ACC ATC TCT GGA CAC GGT GGC TTA GT-3′ |
| | (SEQ ID NO: 5) |
| KH1A02 | 5′-ATC CAG AGT GAC GCA GCA GGC ATA GAT GTG CAG CTC CAA |
| | GGA GAA GAA GGA GTT CTG TGT ATT GGA CAC GGT GGC TTA GT-3′ |
| | (SEQ ID NO: 6) |
| KH1C12 | 5′-ATC CAG AGT GAC GCA GCA TGC CCT AGT TAC TAC TAC TCT TTT |
| | TAG CAA ACG CCC TCG CTT TGG ACA CGG TGG CTT AGT-3′ |
| | (SEQ ID NO: 7) |
| TLS11a | 5′-ACA GCA TCC CCA TGT GAA CAA TCG CAT TGT GAT TGT TAC |
| | GGT TTC CGC CTC ATG GAC GTG CTG-3′ |
| | (SEQ ID NO: 8) |
| PP3 | 5′-ATC CAG AGT GAC GCA GCA CGA GCC AGA CAT CTC ACA CCT |
| | GTT GCA TAT ACA TTT TGC ATG GAC ACG GTG GCT TAG T-3′ |
| | (SEQ ID NO: 9) |
| TV02 | 5′-ATC GTC TGC TCC GTC CAA TAC CTG CAT ATA CAC TTT GCA |
| | TGT GGT TTG GTG TGA GGT CGT GC-3′ |
| | (SEQ ID NO: 10) |
| HCH07 | 5′-TAC CAG TGC GAT GCT CAG GCC GAT GTC AAC TTT TTC TAA CTC |
| | ACT GGT TTT GCC TGA CGC ATT CGG TTG AC-3′ |
| | (SEQ ID NO: 11) |
| KDED2a-3 | 5′-TGC CCG CGA AAA CTG CTA TTA CGT GTG AGA GGA AAG ATC |
| | ACG CGG GTT CGT GGA CAC GG-3′ |
| | (SEQ ID NO: 12) |
| KCHA10 | 5′-ATC CAG AGT GAC GCA GCA GGG GAG GCG AGA GCG CAC AAT |
| | AAC GAT GGT TGG GAC CCA ACT GTT TGG ACA CGG TGG CTT AGT-3′ |
| | (SEQ ID NO: 13) |
| S11e | 5′-ATG CGA ACA GGT GGG TGG GTT GGG TGG ATT GTT CGG CTT CTT |
| | GAT-3′ |
| | (SEQ ID NO: 14) |
| DOV4 | 5′-ACT CAA CGA ACG CTG TGG AGG GCA TCA GAT TAG GAT CTA |
| | TAG GTT CGG ACA TCG TGA GGA CCA GGA GAG CA-3′ |
| | (SEQ ID NO: 15) |
| aptTOV1 | 5′-ATC CAG AGT GAC GCA GCA GAT CTG TGT AGG ATC GCA GTG |
| | TAG TGG ACA TTT GAT ACG ACT GGC TCG ACA CGG TGG CTT A-3′ |
| | (SEQ ID NO: 16) |
| KMF2-1a | 5′-AGG CGG CAG TGT CAG AGT GAA TAG GGG ATG TAC AGG TCT |
| | GCA CCC ACT CGA GGA GTG ACT GAG CGA CGA AGA CCC C-3′ |
| | (SEQ ID NO: 17) |
| EJ2 | 5′-AGT GGT CGA ACT ACA CAT CCT TGA ACT GCG GAA TTA TCT AC-3′ |
| | (SEQ ID NO: 18) |
| CSC01 | 5′-ACC TTG GCT GTC GTG TTG TAG GTG GTT TGC TGC GGT GGG CTC |
| | AAG AAG AAA GCG CAA AGT CAG TGG TCA GAG CGT-3′ |
| | ((SEQ ID NO: 19) |
| Anti- | 5′-CAC TAC AGA GGT TGC GTC TGT CCC ACG TTG TCA TGG GGG GTT |
| EpCAM | GGC CTG-3′ |
| aptamer | (SEQ ID NO: 20) |
| (SYL3C) | |
| Anti- | 5′-GGC GCU CCG ACC UUA GUC UCU GUG CCG CUA UAA UGC ACG |
| EGFR | GAU UUA AUC GCC GUA GAA AAG CAU GUC AAA GCC GGA ACC GUG |
| aptamer | UAG CAC AGC AGAGAAUUAAAUGCCCGCCAUGACCAG-3′ |
| | (SEQ ID NO: 21) |
| Anti- | 5′-ACCAAGACCUGACUUCUAACUAAGUCUACGUUCC-3′ |
| PSMA | (SEQ ID NO: 22) |
| aptamer | |

### EXAMPLE 6

### Gem Chip for High Efficiency and High Purity Cell Capture

The development of a GEM chip for high-efficiency and high-purity tumor cell capture from pancreatic cancer patients is provided. The release and culture of the captured tumor cells, as well as the isolation of CTCs from cancer patients is also demonstrated. The high-performance microchip is based on geometrically optimized micromixer structures, which enhance the transverse flow and flow folding which maximizes the interaction between CTCs and antibody-coated surfaces. With the optimized channel geometry and flow rate, the capture efficiency reached >90% with a purity of >84% when capturing spiked tumor cells in buffer. The system was further validated by isolating a wide range of spiked tumor cells (50-50,000) in 1 mL of lysed blood and whole blood. With the combination of trypsinization and high flow rate washing, captured tumor cells were efficiently released. The released cells were viable and able to proliferate, and showed no difference compared with intact cells that were not subjected to the capture and release process. Furthermore, we applied the device for detecting CTCs from metastatic pancreatic cancer patients' blood and CTCs were found from 17 out of 18 samples (>94%). Potential utility of the device in monitoring the response to anti-cancer drug treatment in pancreatic cancer patients was also tested and the CTC numbers correlated with the clinical computed tomograms (CT scans) of tumors. Accordingly, this embodiment of the present invention provides accurate CTC enumeration, biological studies of CTCs and cancer metastasis, as well as cancer diagnosis and treatment monitoring.

Pancreatic cancer is the fourth leading cause of cancer deaths in the United States, with the poorest 5-year survival rate (6%) for all cancer stages. Over 90% of pancreatic cancers progress to become metastatic. The poor prognosis of pancreatic cancer patients is related to the early dissemination of the disease and the lack of early detection. As discussed above, CTCs can be used to track metastasis, cancer diagnosis and monitoring cancer status. While biopsy is the current gold standard of cancer diagnosis, it involves removal of tissues or cells from the body and examination by experienced surgeons and pathologists. The invasive nature of biopsy prevents patients from being tested in an ongoing or repetitive basis. CTC examination, on the other hand, is much less invasive, with only 5-10 mL of patient blood needed; it is like a blood test for cancer. CTC monitoring is regarded as “liquid biopsy” or “live biopsy” of a tumor, which enables noninvasive cancer diagnosis and real-time monitoring of therapeutic response.

Staggered herringbone micromixers have been developed for fluid mixing in microchannels and have been exploited for enhancing the cell capture. Yet, limited research has been reported on the optimization of herringbone mixers for high-performance cell capture. Different from mixing solutions through transverse flow, inducing cell-surface interactions requires cells with nearly zero diffusivity for advection to microchannel surface. Herein, we have developed a GEM chip for high-performance CTC capture (high efficiency, purity, throughput and cell viability). With experimental optimization of the herringbone micromixers, we achieved capture of spiked tumor cells with >90% capture efficiency and >84% purity. In addition, the time required to process 1 mL blood sample is <17 min, much faster than those reported in literature. Since very limited work has been done on cellular studies after CTC capture, we have investigated the release, the viability and the culture of the captured cells. Captured cells can be efficiently released with the combined methods of trypsinization and high flow rate washing. Experiments also showed that in culture the released cells grew as well as intact cells that had not been subjected to the capture and release process. Further, we applied the device for isolation of CTCs from pancreatic cancer patients, with CTCs observed in 17 of 18 patient samples. We also demonstrated the potential of using CTC enumeration as a surrogate for radiographic monitoring of chemotherapy response in pancreatic cancer patients. Our device sensitivity enables isolation and enumeration of CTCs from pancreatic cancer patients, a disease where invasive biopsies are difficult and the commercial CellSearch system has proven to be inefficient. Compared with reported efforts, this work demonstrated a systematic study of the following aspects: geometric optimization of a micromixer for enhanced target CTC capture, release and re-culture of captured tumor cells, cell viability before and after release, cell binding behaviors after release and re-culture, isolation and counting of understudied pancreatic CTCs, comparison of CTC enumeration with CT scans for monitoring chemotherapy response in pancreatic cancer patients. A comprehensive study of these aspects would further improve CTC isolation performance help understand post-capture processing of CTCs and push forward CTC isolation for cancer diagnosis. A comparison of this work with published studies is detailed in Table 3.

TABLE 3
Comparison of this work with those in the literature.

| | | Capture | | | Cell | | |
|---|---|---|---|---|---|---|---|
| Reference | Device | efficiency | Purity* | Throughput* | viability | Release | Culture |
| 1 | CTC chip | 65% | ~50% | 0.5-1 h/mL | 98.5 ± 2.3% | No | No |
| 2 | HB-chip | ~91.8% | Higher | ~0.83 h/mL | 95% ± 0.6% | No | No |
| | | | than [1] | | | | |
| 3 | Sinusoidal | >97% | NA* | ~0.5 h/mL | NA | Yes | No |
| | channel | | | | | | |
| 4 | GEDI | 85-97% | 68 ± 6% | 1 h/mL | NA | No | No |
| | chip | | | | | | |
| 5 | 3D- | >95% | NA | 1 h/mL | 84-91% | No | No |
| | nanopillar | | | | | | |
| | & Mixer | | | | | | |
| This work | GEM chip | >90% | ~84% | 0.28 h/mL | ~89% | Yes | Yes |

Note*:
1) NA indicates “not available” or “not applicable”. 2) Throughput is determined by the flow rate, which is inversely proportional to the time required to process sufficient amount of sample that contains detectable number of CTCs. The time required to process 1 mL of sample is listed. 3) Purity varies with the target/control cell ratio and depends on whether obtained from buffer system or whole blood; thus purity here is just for reference not for comparison.

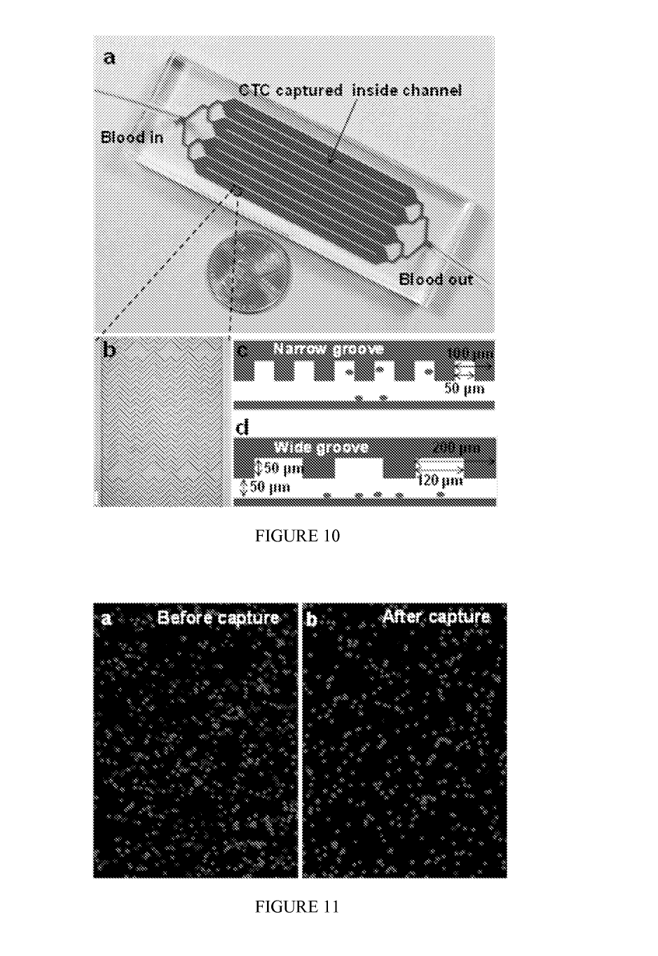

In this study, we first developed a geometrically enhanced mixing chip (GEM chip) based on patterned herringbone or chevron structures. The mixer design was inspired by several groups, and the dimensions were optimized for high-efficiency and high-purity cell capture. As shown in FIG. 10, the GEM chip is the same size as a microscope slide (3 in.×1 in.), having 8 parallel channels with uniform flow to form a high throughput device. Each channel is 2.1-mm wide, 50-μm deep, with 50-μm deep herringbone grooves repeating over a total length of 50 mm. The staggered herringbone grooves disrupt streamlines and induce chaotic mixing and microvortex, which maximize collisions and interactions between target cells and device surfaces, leading to increased cell capture efficiency. The groove width and the groove pitch were carefully selected for high-performance cell capture, as discussed below.

Target Cell Capture from a Homogenous Cell Mixture

The performance of the device was first evaluated by sorting a mixture of pancreatic cancer cell lines: target L3.6pl cells (EpCAM+) and control MIA PaCa-2 cells (EpCAM-). Flow cytometry results show that L3.6pl cells bind strongly with anti-EpCAM, while MIA PaCa-2 cells do not bind with anti-EpCAM (FIGS. 20 and 22). This indicates that L3.6pl cells express a significant number of EpCAM receptors, while MIA PaCa-2 cells express negligible surface EpCAM, which is consistent with data already reported in literature. To start the cell capture, biotinylated anti-EpCAM was first immobilized on the surface of microchannel. Then a cell mixture containing 10⁶ L3.6pl cells (stained with Vybrant DiI, red) and 10⁶ MIA PaCa-2 cells (stained with Vybrant DiD, blue) per mL sample was introduced into the microchannel. FIG. 11a shows a representative image of the cell mixture prior to sorting, with same number of target cells and control cells. FIG. 11b shows a typical image after the cell mixture was processed through the device, with L3.6pl cells in the majority, while most control MIA PaCa-2 cells were removed by washing. FIG. 11a indicates that significant enrichment of target cells can be achieved using the antibody-coated microfluidic device.

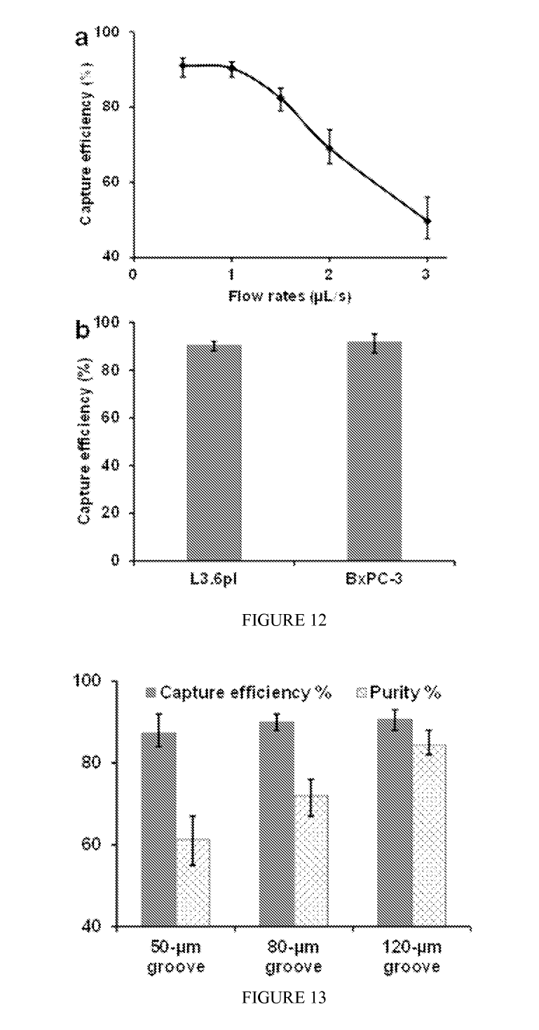

After the initial experiments, different flow rates were used to study the effects of flow rate on cell capture efficiency. As shown in FIG. 12a, the capture efficiency of L3.6pl cells was >90% at low flow rates, but decreased dramatically at flow rates above 1 μL/s, primarily due to the reduced interaction time between the cells and antibody-coated surfaces as well as the increased shear stress at higher flow rates. To obtain both efficient capture and sufficient throughput, an optimal flow rate of 1 μL/s was chosen, with a flow velocity of 0.75 mm/s and maximum shear stress of 0.38 dyn/cm² at the wall. As shown in FIG. 12b, the capture efficiency was (90±2) % for L3.6pl cells and (92±4) % for BxPC-3 cells at 1 μL/s.

Micromixer device optimization for high-performance cell capture

When traditional micromixer design dimensions were used (HB chip, FIG. 10c) for pancreatic tumor cell capture, we found that non-target cells were easily trapped in the device (causing low CTC capture purity) and cells were not captured on the same focus plane (making imaging and counting difficult). This may be because cell trapping took place in narrow grooves (with high aspect ratio) as illustrated in FIG. 10c, and an increased groove width would give better purity. Thus two new designs were made by increasing the groove width from 50 μm (narrow groove, FIGS. 10c) to 80 μm and 120 μm (wide groove, FIG. 10d). Experimental results proved that a wider groove with increased groove pitch achieved high purity cell capture, while maintaining cell capture efficiency. As shown in FIG. 13, with a groove width of 120 μm a capture purity of 84% was obtained, while the traditional 50-μm groove width yielded only 61% purity. In addition, the capture efficiency for the wide groove design was not reduced and may have increased slightly, which agrees with simulation study by Forbes et al.

Tumor Cell Capture from Lysed Blood and Whole Blood

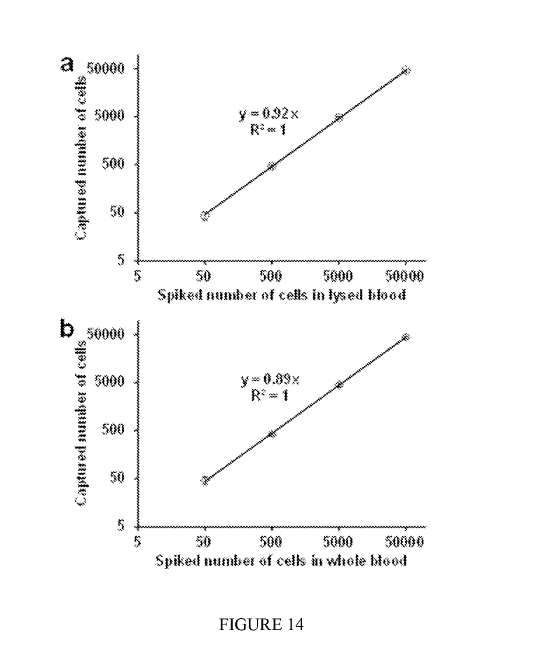

To test cell capture under more physiological conditions and to mimic CTC capture from patient blood, a series of experiments in which labeled L3.6pl cells were spiked in lysed or whole blood were performed. Samples were prepared by spiking 50-50,000 L3.6pl cells in 1 mL lysed blood or whole blood. After being pumped through the micromixer device, as many as ˜92% of L3.6pl cells were captured from lysed blood (FIGS. 14a), and ˜89% of L3.6pl cells were captured from whole blood (FIG. 14b), proving that the device and the conditions are suitable for capturing CTCs from patient blood specimens with or without prior red blood cell lysis.

Cell Release and Cell Viability

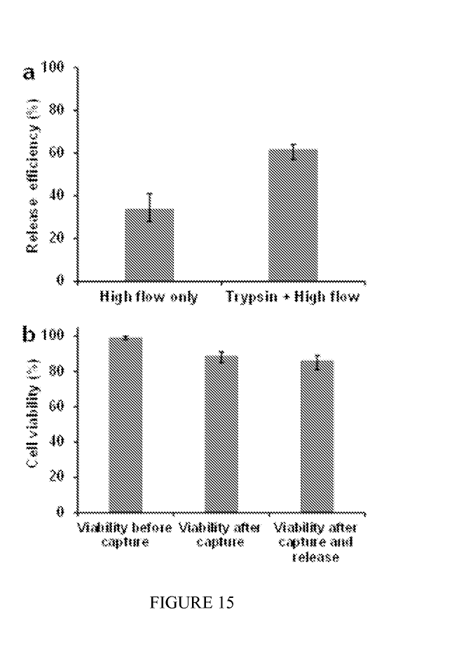

The detachment and release of captured cells in antibody-coated microchannels was achieved by using a combination of trypsinization (enzymatic release) and high flow rate washing (high shear stress). Detached cells were collected in a cell culture dish with fresh medium for propagation in cell culture. As shown in FIG. 15a, the release efficiency of L3.6pl cells increased to >60% by using the combined releasing method, while high flow washing alone gave only ˜30% release. Trypsin release and shear stress-based release procedures cause minimum cell damage as proved by cell viability assay and flow cytometry. PI/AO assay was used to test the viability of released cells, with >85% cells remaining viable after the capturing and release process (FIG. 15b), making the isolated tumor cells suitable for subsequent cellular analysis. Flow cytometry tests also showed that released L3.6pl cells retain their binding with anti-EpCAM, as shown in FIG. 23b.

Re-Culture of Captured Cells

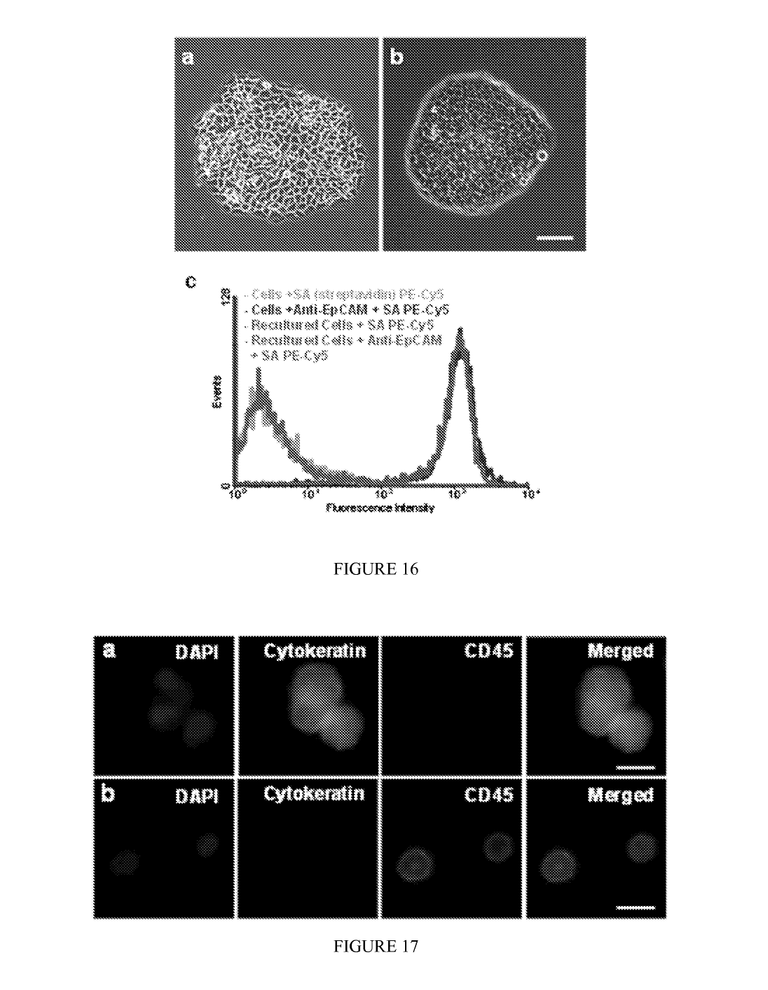

To determine whether isolated tumor cells can be cultured, 5,000 L3.6pl cells were spiked into whole blood and subjected to the capture and release process as discussed above. The released cells were then seeded into cell culture dishes for propagation in culture. As a comparison, 5,000 intact L3.6pl cells (not subjected to the culture and release process) were directly seeded for culture with the same conditions. Results showed that both adhered well and proliferated on the culture dishes forming large clusters and colonies by day 9 (shown in FIG. 16) and growth to confluence with longer time (14 days), although the captured cells took a little longer to reach confluence than intact cells. Then we were able to trypsinize these cells and seed them to other culture dishes, where they grew as adherent monolayers. The isolated cells have successfully undergone multiple (>8) passages without loss of viability or detectable changes in behavior. Flow cytometry tests indicated that the isolated cells maintain binding behavior with anti-EpCAM, as shown in FIG. 16c. These results clearly demonstrate that tumor cell lines isolated from whole blood retain both their viability and their proliferation ability, which are crucial for CTC cellular analysis.

### EXAMPLE 7

### Isolation of CTCS from Patients with Pancreatic Cancer Using Gem Chip

Blood samples from patients with metastatic pancreatic cancer (stage IV) were analyzed for CTC enumeration using the device and conditions described in Example 6. Since EpCAM has been known to be overexpressed in pancreatic adenocarcinoma, anti-EpCAM was used as the capture agent. Milliliters of patient blood were pumped through the antibody-coated device. After fixation and permeabilization, three-color immunocytochemistry was utilized to identify and count CTCs from nonspecifically captured white blood cells, using FITC-labeled anti-Cytokeratin (CK, green), PE-labeled anti-CD45 (red) and DAPI (blue) for staining As shown in FIG. 17, CTCs are DAPF/CK+/CD45− cells, while WBCs are DAPI+/CK-/CD45+ cells. A significant population of “double positive” cells with both hematopoietic and epithelial markers (CK+/CD45+) were found in quite a few patient samples (average ˜2 “double positive” cells in 1 mL patient blood). Since the origin and significance of these cells are under debate, we temporarily excluded them from CTC counting. However, detailed numbers of “double positive” cells were presented in Table 4. For the 18 pancreatic cancer patient samples processed, CTCs were found in 17 cases (>94%), with an average number of 3 CTCs per mL of blood, as shown in Table 4. To examine the possibility of false positives, we investigated capturing CTCs from whole blood of normal healthy individuals. Similar volumes of blood were run through our device using the same protocol. Table 5 shows the results from blood samples of nine healthy donors. Zero CTCs were detected from blood samples of all normal healthy individuals studied, thus showing a false positive rate of zero. Additionally, we found much fewer “double positive” cells in healthy donors' blood than in patient blood, indicating that most of the “double positive” cells could be the heterogeneous CTCs or the nonspecific binding of anti-CD45 to CTCs. Further studies with additional markers are required to understand and explain these “double positive” cells.

TABLE 4
Quantification of CTCs and “double positive” cells per mL of blood
among 18 samples from patients with metastatic pancreatic cancer.

| | | Volume | Raw | | “Double |
|---|---|---|---|---|---|
| Sample | Cancer | processed | number of | | positive” |
| No. | type | (mL) | CTCs | CTCs/mL | cells/mL |
| 1 | Pancreas | 2 | 4 | 2 | 1 |
| 2 | Pancreas | 4 | 14 | 4 | 0 |
| 3 | Pancreas | 2 | 9 | 5 | 3 |
| 4 | Pancreas | 1 | 2 | 2 | 6 |
| 5 | Pancreas | 2 | 2 | 1 | 1 |
| 6 | Pancreas | 2 | 0 | 0 | 2 |
| 7 | Pancreas | 2 | 5 | 3 | 4 |
| 8 | Pancreas | 1 | 2 | 2 | 0 |
| 9 | Pancreas | 2 | 4 | 2 | 5 |
| 10 | Pancreas | 4 | 19 | 5 | 2 |
| 11 | Pancreas | 2 | 5 | 3 | 0 |
| 12 | Pancreas | 2 | 4 | 2 | 0 |
| 13 | Pancreas | 4 | 5 | 1 | 1 |
| 14 | Pancreas | 4 | 15 | 4 | 3 |
| 15 | Pancreas | 4 | 16 | 4 | 2 |
| 16 | Pancreas | 4 | 29 | 7 | 4 |
| 17 | Pancreas | 2 | 6 | 3 | 1 |
| 18 | Pancreas | 2 | 2 | 1 | 0 |

TABLE 5
Quantification of CTCs in healthy donor blood.

| | Healthy Sample | | | | | | | | |
|---|---|---|---|---|---|---|---|---|---|
| | 1 | 2 | 3 | 4 | 5 | 6 | 7 | 8 | 9 |
| Number | 0 | 0 | 0 | 0 | 0 | 0 | 0 | 0 | 0 |
| of CTCs | | | | | | | | | |

For capturing CTCs from patient blood, much more capture of leukocytes was observed than the spiking experiments using healthy donor's blood. For 1 mL of blood processed, ˜3500 leukocytes were captured for spiking experiments using healthy samples, while >24000 of leukocytes were captured for patient samples. This could due to complexity of patient blood conditions. The high purity of the GEM chip shows advantages over the traditional mixing chip when enumerating patient CTCs. The GEM chip would have been able to detect an average of ˜23 CTCs from 7.5 mL blood, much higher than the cut-off number of CellSearch system. Considering that CellSearch is inefficient for pancreatic cancer, the GEM chip of this embodiment of the current invention provides a powerful tool for CTC enumeration in pancreatic cancer. In addition, with a flow rate of 1 μL/s (3.6 mL/h), 1 mL blood sample can be processed within 17 min, which gives sufficient throughput for clinical applications.

Monitoring Anti-Cancer Treatment Response Using CTCs

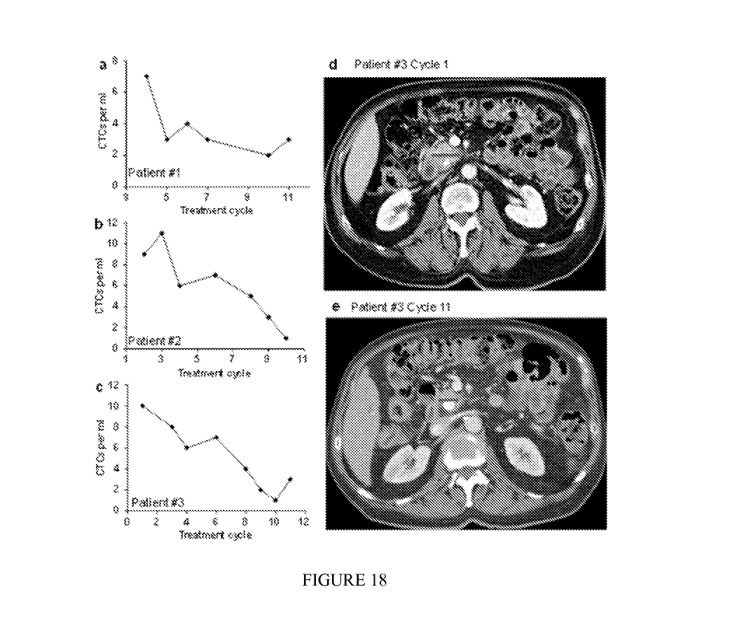

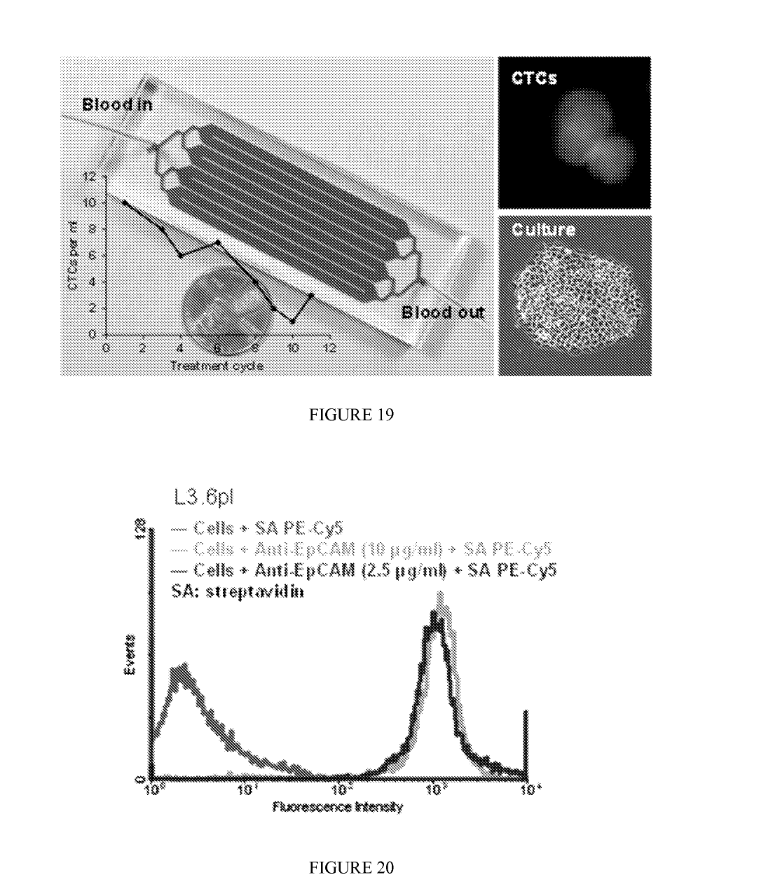

To demonstrate the unique clinical potential of device and system provided by certain embodiments of the current invention, the relation between the CTC number and tumor size in patients with pancreatic cancer undergoing chemotherapy was tested. Three patients with stage IV metastatic pancreatic cancer (deemed unresectable) were included in the analysis. Each patient received identical standard treatments with the identical palliative chemotherapy and with X-ray computed tomography (CT) scans done at the same intervals. Blood samples were collected at baseline and at the first day of each subsequent treatment cycle. CTCs were captured and counted using the device and methods discussed above. Investigators were blinded to the demographic and clinicopathological characteristics of the patients. The number of CTCs captured at different treatment cycles is plotted in FIG. 18a-c. In general, the CTC number decreased with continuation of treatment and modeled the CT scan results (which represent standard clinical response measurements). The CTC number correlated proportionally with CT scan-measured tumor size in each of the three patients. FIGS. 18d & 18e show that tumor size decreased as treatment progressed for patient #3, which was reflected by the trend of CTC number in FIG. 18c. CT scan data from patient #1 and patient #2 also indicated either reduced primary tumor size or reduced metastatic tumor burden (data not shown). Together, these results indicate that CTC quantification using our device correlates with clinical response and findings from CT imaging, but causes significantly less harms to patients than standard clinical radiographic measurements. The noninvasive nature of the devices and methods of the current invention provides a powerful tool for monitoring early response or failure to cancer treatment and potentially early cancer diagnosis and relapse prediction.

As such, this embodiment of the current invention demonstrates an efficient CTC capture platform based on a GEM chip. The device achieved >90% capture efficiency, >84% purity with a throughput of processing 3.6 mL blood in 1 hour. The system was then utilized to isolate CTCs from pancreatic cancer patient blood samples, with CTCs detected in 17 of 18 samples. We also successfully demonstrated positive correlation in monitoring anti-cancer treatment response using the CTC numbers obtained from certain devices of the current invention. In addition, the captured cells were released from the devices described in this example with >61% release efficiency, and with >86% viability. Furthermore, the ability to culture the captured cells, a critical requirement for post-isolation cellular analysis, is also demonstrated. Although it is extremely challenging to culture the isolated CTCs from a patient's blood and to develop a new cell line, the devices and methods of certain embodiments of the current invention show the possibility to culture spiked tumor cells, after the sophisticated capture and release process, while maintaining their viability and proliferation capability. Therefore, CTC capture system of this example of the current invention shows great potential for efficient CTC enrichment, isolation, and cellular/genetic analysis, leading to now feasible “liquid biopsy” of pancreatic cancer.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Kling, J., Beyond counting tumor cells. Nat Biotech 2012, 30, 578-580.
2. Yu, M.; Stott, S.; Toner, M.; Maheswaran, S.; Haber, D. A., Circulating tumor cells:

approaches to isolation and characterization. The Journal of Cell Biology 2011, 192, 373-382.
3. Galanzha, E. I.; Shashkov, E. V.; Kelly, T.; Kim, J.-W.; Yang, L.; Zharov, V. P., In vivo magnetic enrichment and multiplex photoacoustic detection of circulating tumour cells. Nat Nano 2009, 4, 855-860.
4. Pantel, K.; Brakenhoff, R. H.; Brandt, B., Detection, clinical relevance and specific biological properties of disseminating tumour cells. Nat. Rev. Cancer 2008, 8, 329-340.
5. Cristofanilli, M.; Budd, G. T.; Ellis, M. J.; Stopeck, A.; Matera, J.; Miller, M. C.; Reuben, J. M.; Doyle, G. V.; Allard, W. J.; Terstappen, L. W. M. M., et al., Circulating Tumor Cells, Disease Progression, and Survival in Metastatic Breast Cancer. N. Engl. J. Med. 2004, 351, 781-791.
6. Danila, D. C.; Fleisher, M.; Scher, H. I., Circulating Tumor Cells as Biomarkers in Prostate Cancer. Clin. Cancer. Res. 2011, 17, 3903-3912.
7. He, W.; Wang, H.; Hartmann, L. C.; Cheng, J.-X.; Low, P. S., In vivo quantitation of rare circulating tumor cells by multiphoton intravital flow cytometry. Proceedings of the National Academy of Sciences 2007, 104, 11760-11765.
8. Issadore, D.; Chung, J.; Shao, H.; Liong, M.; Ghazani, A. A.; Castro, C. M.; Weissleder, R.; Lee, H., Ultrasensitive Clinical Enumeration of Rare Cells ex Vivo Using a Micro-Hall Detector. Sci. Transl. Med. 2012, 4, 141ra92.
9. Riethdorf, S.; Fritsche, H.; Muller, V.; Rau, T.; Schindlbeck, C.; Rack, B.; Janni, W.; Coith, C.; Beck, K.; Jänicke, F., et al., Detection of Circulating Tumor Cells in Peripheral Blood of Patients with Metastatic Breast Cancer: A Validation Study of the CellSearch System. Clin. Cancer. Res. 2007, 13, 920-928.
10. Balic, M.; Williams, A.; Lin, H.; Datar, R.; Cote, R. J., Circulating Tumor Cells: From Bench to Bedside. Annu. Rev. Med. 2013, 64, 31-44.
11. Nagrath, S.; Sequist, L. V.; Maheswaran, S.; Bell, D. W.; Irimia, D.; Ulkus, L.; Smith, M. R.; Kwak, E. L.; Digumarthy, S.; Muzikansky, A., et al., Isolation of rare circulating tumour cells in cancer patients by microchip technology. Nature 2007, 450, 1235-1239.
12. Stott, S. L.; Hsu, C.-H.; Tsukrov, D. I.; Yu, M.; Miyamoto, D. T.; Waltman, B. A.;

Rothenberg, S. M.; Shah, A. M.; Smas, M. E.; Korir, G. K., et al., Isolation of circulating tumor cells using a microvortex-generating herringbone-chip. Proceedings of the National Academy of Sciences 2010.
13. Ozkumur, E.; Shah, A. M.; Ciciliano, J. C.; Emmink, B. L.; Miyamoto, D. T.;

Brachtel, E.; Yu, M.; Chen, P.-i.; Morgan, B.; Trautwein, J., et al., Inertial Focusing for Tumor Antigen-Dependent and -Independent Sorting of Rare Circulating Tumor Cells. Sci. Transl. Med. 2013, 5, 179ra47.
14. Schiro, P. G.; Zhao, M.; Kuo, J. S.; Koehler, K. M.; Sabath, D. E.; Chiu, D. T.,

Sensitive and High-Throughput Isolation of Rare Cells from Peripheral Blood with Ensemble-Decision Aliquot Ranking Angew. Chem. Int. Ed. 2012, 51, 4618-4622.
15. Zheng, X.; Cheung, L. S.-L.; Schroeder, J. A.; Jiang, L.; Zohar, Y., A high-performance microsystem for isolating circulating tumor cells. Lab Chip 2011.
16. Phillips, J. A.; Xu, Y.; Xia, Z.; Fan, Z. H.; Tan, W., Enrichment of Cancer Cells Using Aptamers Immobilized on a Microfluidic Channel. Anal. Chem. 2009, 81, 1033-1039.
17. Xu, Y.; Phillips, J. A.; Yan, J.; Li, Q.; Fan, Z. H.; Tan, W., Aptamer-Based Microfluidic Device for Enrichment, Sorting, and Detection of Multiple Cancer Cells. Anal. Chem. 2009, 81, 7436-7442.
18. Sheng, W.; Chen, T.; Kamath, R.; Xiong, X.; Tan, W.; Fan, Z. H., Aptamer-Enabled Efficient Isolation of Cancer Cells from Whole Blood Using a Microfluidic Device. Anal. Chem. 2012, 84, 4199-4206.
19. Gleghorn, J. P.; Pratt, E. D.; Denning, D.; Liu, H.; Bander, N. H.; Tagawa, S. T.;

Nanus, D. M.; Giannakakou, P. A.; Kirby, B. J., Capture of circulating tumor cells from whole blood of prostate cancer patients using geometrically enhanced differential immunocapture (GEDI) and a prostate-specific antibody. Lab Chip 2010, 10, 27-29.
20. Adams, A. A.; Okagbare, P. I.; Feng, J.; Hupert, M. L.; Patterson, D.; Gottert, J.; McCarley, R. L.; Nikitopoulos, D.; Murphy, M. C.; Soper, S. A., Highly Efficient Circulating Tumor Cell Isolation from Whole Blood and Label-Free Enumeration Using Polymer-Based Microfluidics with an Integrated Conductivity Sensor. J. Am. Chem. Soc. 2008, 130, 8633-8641.
21. Wang, S.; Wang, H.; Jiao, J.; Chen, K.-J.; Owens, G. E.; Kamei, K.-i.; Sun, J.; Sherman, D. J.; Behrenbruch, C. P.; Wu, H., et al., Three-Dimensional Nanostructured Substrates toward Efficient Capture of Circulating Tumor Cells. Angew. Chem. Int. Ed. 2009, 48, 8970-8973.
22. Wang, S.; Liu, K.; Liu, J.; Yu, Z. T. F.; Xu, X.; Zhao, L.; Lee, T.; Lee, E. K.; Reiss, J.; Lee, Y.-K., et al., Highly Efficient Capture of Circulating Tumor Cells by Using Nanostructured Silicon Substrates with Integrated Chaotic Micromixers. Angew. Chem. Int. Ed. 2011, 50, 3084-3088.
23. Mammen, M.; Choi, S.-K.; Whitesides, G. M., Polyvalent Interactions in Biological Systems: Implications for Design and Use of Multivalent Ligands and Inhibitors. Angew. Chem. Int. Ed. 1998, 37, 2754-2794.
24. Kitov, P. I.; Sadowska, J. M.; Mulvey, G.; Armstrong, G. D.; Ling, H.; Pannu, N. S.;

Read, R. J.; Bundle, D. R., Shiga-like toxins are neutralized by tailored multivalent carbohydrate ligands. Nature 2000, 403, 669-672.
25. de la Fuente, J. M.; Barrientos, A. G.; Rojas, T. C.; Rojo, J.; Canada, J.; Fernandez, A.; Penadés, S., Gold Glyconanoparticles as Water-Soluble Polyvalent Models To Study Carbohydrate Interactions. Angew. Chem. 2001, 113, 2317-2321.
26. Weissleder, R.; Kelly, K.; Sun, E. Y.; Shtatland, T.; Josephson, L., Cell-specific targeting of nanoparticles by multivalent attachment of small molecules. Nat. Biotechnol. 2005, 23, 1418-23.
27. Fasting, C.; Schalley, C. A.; Weber, M.; Seitz, O.; Hecht, S.; Koksch, B.; Dernedde, J.; Graf, C.; Knapp, E.-W.; Haag, R., Multivalency as a Chemical Organization and Action Principle. Angew. Chem. Int. Ed. 2012, 51, 10472-10498.
28. Massich, M. D.; Giljohann, D. A.; Schmucker, A. L.; Patel, P. C.; Mirkin, C. A., Cellular Response of Polyvalent Oligonucleotide—Gold Nanoparticle Conjugates. ACS Nano 2010, 4, 5641-5646.
29. Kim, Y.; Cao, Z.; Tan, W., Molecular assembly for high-performance bivalent nucleic acid inhibitor. Proceedings of the National Academy of Sciences 2008, 105, 5664-5669.
30. Zhao, W.; Cui, C. H.; Bose, S.; Guo, D.; Shen, C.; Wong, W. P.; Halvorsen, K.; Farokhzad, O. C.; Teo, G. S.; Phillips, J. A., et al., Bioinspired multivalent DNA network for capture and release of cells. Proc. Natl. Acad. Sci. U.S.A. 2012, 109, 19626-31.
31. Hong, S.; Leroueil, P. R.; Majoros, I. J.; Orr, B. G.; Baker Jr, J. R.; Banaszak Holl, M. M., The Binding Avidity of a Nanoparticle-Based Multivalent Targeted Drug Delivery Platform. Chemistry & amp; Biology 2007, 14, 107-115.
32. Myung, J. H.; Gajjar, K. A.; Saric, J.; Eddington, D. T.; Hong, S., Dendrimer-Mediated Multivalent Binding for the Enhanced Capture of Tumor Cells. Angew. Chem. Int. Ed. 2011, 50, 11769-11772.
33. Huang, Y.-F.; Chang, H.-T.; Tan, W., Cancer Cell Targeting Using Multiple Aptamers Conjugated on Nanorods. Anal. Chem. 2008, 80, 567-572.
34. Pavlov, V.; Xiao, Y.; Shlyahovsky, B.; Willner, I., Aptamer-Functionalized Au Nanoparticles for the Amplified Optical Detection of Thrombin. J. Am. Chem. Soc. 2004, 126, 11768-11769.
35. Farokhzad, 0. C.; Jon, S.; Khademhosseini, A.; Tran, T.-N. T.; LaVan, D. A.; Langer, R., Nanoparticle-Aptamer Bioconjugates. Cancer Res. 2004, 64, 7668-7672.
36. Shangguan, D.; Li, Y.; Tang, Z.; Cao, Z. C.; Chen, H. W.; Mallikaratchy, P.; Sefah, K.; Yang, C. J.; Tan, W., Aptamers evolved from live cells as effective molecular probes for cancer study. Proceedings of the National Academy of Sciences 2006, 103, 11838-11843.
37. Keefe, A. D.; Pai, S.; Ellington, A., Aptamers as therapeutics. Nat Rev Drug Discov 2010, 9, 537-550.
38. Wang, H.; Yang, R.; Yang, L.; Tan, W., Nucleic Acid Conjugated Nanomaterials for Enhanced Molecular Recognition. ACS Nano 2009, 3, 2451-2460.
39. Zheng, D.; Seferos, D. S.; Giljohann, D. A.; Patel, P. C.; Mirkin, C. A., Aptamer Nano-flares for Molecular Detection in Living Cells. Nano Lett. 2009, 9, 3258-3261.
40. Chen, T.; Shukoor, M. I.; Wang, R.; Zhao, Z.; Yuan, Q.; Bamrungsap, S.; Xiong, X.; Tan, W., Smart Multifunctional Nanostructure for Targeted Cancer Chemotherapy and Magnetic Resonance Imaging. ACS Nano 2011, 5, 7866-7873.
41. Bamrungsap, S.; Chen, T.; Shukoor, M. I.; Chen, Z.; Sefah, K.; Chen, Y.; Tan, W., Pattern Recognition of Cancer Cells Using Aptamer-Conjugated Magnetic Nanoparticles. ACS Nano 2012, 6, 3974-3981.
42. Mirkin, C. A.; Letsinger, R. L.; Mucic, R. C.; Storhoff, J. J., A DNA-based method for rationally assembling nanoparticles into macroscopic materials. Nature 1996, 382, 607-609.
43. Cutler, J. I.; Auyeung, E.; Mirkin, C. A., Spherical nucleic acids. J. Am. Chem. Soc. 2012, 134, 1376-91.
44. Chen, W.; Weng, S.; Zhang, F.; Allen, S.; Li, X.; Bao, L.; Lam, R. H.; Macoska, J. A.; Merajver, S. D.; Fu, J., Nanoroughened Surfaces for Efficient Capture of Circulating Tumor Cells without Using Capture Antibodies. ACS Nano 2013, 7, 566-75
45. Lee, S.-K.; Kim, G.-S.; Wu, Y.; Kim, D.-J.; Lu, Y.; Kwak, M.; Han, L.; Hyung, J.-H.; Seol, J.-K.; Sander, C., et al., Nanowire Substrate-Based Laser Scanning Cytometry for Quantitation of Circulating Tumor Cells. Nano Lett. 2012, 12, 2697-2704.
46. Hurst, S. J.; Lytton-Jean, A. K. R.; Mirkin, C. A., Maximizing DNA Loading on a Range of Gold Nanoparticle Sizes. Anal. Chem. 2006, 78, 8313-8318.
47. Tang, Z.; Shangguan, D.; Wang, K.; Shi, H.; Sefah, K.; Mallikratchy, P.; Chen, H. W.; Li, Y.; Tan, W., Selection of Aptamers for Molecular Recognition and Characterization of Cancer Cells. Anal. Chem. 2007, 79, 4900-4907.
48. Stroock, A. D.; Dertinger, S. K. W.; Ajdari, A.; Mezié, I.; Stone, H. A.; Whitesides, G. M., Chaotic Mixer for Microchannels. Science 2002, 295, 647-651.
49. Chen, L.; Liu, X.; Su, B.; Li, J.; Jiang, L.; Han, D.; Wang, S., Aptamer-Mediated Efficient Capture and Release of T Lymphocytes on Nanostructured Surfaces. Adv. Mater. 2011, 23, 4376-4380.
50. Cave, H.; van der Werff ten Bosch, J.; Suciu, S.; Guidal, C.; Waterkeyn, C.; Otten, J.; Bakkus, M.; Thielemans, K.; Grandchamp, B.; Vilmer, E., et al., Clinical Significance of Minimal Residual Disease in Childhood Acute Lymphoblastic Leukemia. N. Engl. J. Med. 1998, 339, 591-598.
51. Bottcher, S.; Ritgen, M.; Fischer, K.; Stilgenbauer, S.; Busch, R. M.; Fingerle-Rowson, G.; Fink, A. M.; Buhler, A.; Zenz, T.; Wenger, M. K., et al., Minimal Residual Disease Quantification Is an Independent Predictor of Progression-Free and Overall Survival in Chronic Lymphocytic Leukemia: A Multivariate Analysis From the Randomized GCLLSG CLL8 Trial. J. Clin. Oncol. 2012, 30, 980-988.
52. Valencia, P. M.; Farokhzad, O. C.; Karnik, R.; Langer, R., Microfluidic technologies for accelerating the clinical translation of nanoparticles. Nat. Nanotechnol. 2012, 7, 623-9.
53. Liu, J.; Lu, Y., Preparation of aptamer-linked gold nanoparticle purple aggregates for colorimetric sensing of analytes. Nat. Protocols 2006, 1, 246-252.
54. Zhang, X.; Servos, M. R.; Liu, J., Instantaneous and Quantitative Functionalization of Gold Nanoparticles with Thiolated DNA Using a pH-Assisted and Surfactant-Free Route. J. Am. Chem. Soc. 2012, 134, 7266-7269.
55. Qin, D.; Xia, Y.; Whitesides, G. M., Soft lithography for micro- and nanoscale patterning. Nat. Protocols 2010, 5, 491-502.
56. Forbes, T. P.; Kralj, J. G., Engineering and analysis of surface interactions in a microfluidic herringbone micromixer. Lab Chip 2012, 12, 2634-7.
57. Mata, A.; Fleischman, A. J.; Roy, S., Fabrication of multi-layer SU-8 microstructures. Journal of Micromechanics and Microengineering. 2006, 16, 276.
58. Capretto, L.; Cheng, W.; Hill, M.; and Zhang, X., Micromixing within microfluidic devices, Topics in Current Chemistry, 2011, 304, 27-68.
59. R. Siegel, D. Naishadham and A. Jemal, CA. Cancer J. Clin., 2013, 63, 11-30.
60. K. Tjensvoll, 0. Nordg\dot{a}rd and R. Smaaland, Int. J. Cancer, 2013, n/a-n/a.
61. P. Cen, X. Ni, J. Yang, D. Y. Graham and M. Li, Biochimica et Biophysica Acta (BBA)—Reviews on Cancer, 2012, 1826, 350-356.
62. K. Pantel, C. Alix-Panabieres and S. Riethdorf, Nat Rev Clin Oncol, 2009, 6, 339-351.
63. M. C. Miller, G. V. Doyle and L. W. M. M. Terstappen, Journal of Oncology, 2010, 2010, Article ID 617421.
64. M. A. Eloubeidi, V. K. Chen, I. A. Eltoum, D. Jhala, D. C. Chhieng, N. Jhala, S. M. Vickers and C. M. Wilcox, The American Journal of Gastroenterology, 2003, 98, 2663-2668.
65. E. Crowley, F. Di Nicolantonio, F. Loupakis and A. Bardelli, Nat Rev Clin Oncol, 2013, 10, 472-484.
66. M. Yu, S. Stott, M. Toner, S. Maheswaran and D. A. Haber, The Journal of Cell Biology, 2011, 192, 373-382.
67. W. J. Allard, J. Matera, M. C. Miller, M. Repollet, M. C. Connelly, C. Rao, A. G. J. Tibbe, J. W. Uhr and L. W. M. M. Terstappen, Clin. Cancer. Res., 2004, 10, 6897-6904.
68. X. Zheng, L. S.-L. Cheung, J. A. Schroeder, L. Jiang and Y. Zohar, Lab Chip, 2011, 11, 3269-3276.
69. W. Sheng, T. Chen, W. Tan and Z. H. Fan, ACS Nano, 2013, 7, 7067-7076.
70. J. Chen, J. Li and Y. Sun, Lab Chip, 2012, 12, 1753-1767.
71. S. K. Arya, B. Lim and A. R. A. Rahman, Lab Chip, 2013, 13, 1995-2027.
72. S. L. Stott, C. H. Hsu, D. I. Tsukrov, M. Yu, D. T. Miyamoto, B. A. Waltman, S. M. Rothenberg, A. M. Shah, M. E. Smas, G. K. Korir, F. P. Floyd, A. J. Gilman, J. B. Lord, D. Winokur, S. Springer, D. Irimia, S. Nagrath, L. V. Sequist, R. J. Lee, K. J. Isselbacher, S. Maheswaran, D. A. Haber and M. Toner, Proc. Natl. Acad. Sci. U.S. A., 2010, 107, 18392-18397.
73. J. H. Kang, S. Krause, H. Tobin, A. Mammoto, M. Kanapathipillai and D. E. Ingber, Lab Chip, 2012, 12, 2175-2181.
74. L. Khoja, A. Backen, R. Sloane, L. Menasce, D. Ryder, M. Krebs, R. Board, G. Clack, A. Hughes, F. Blackhall, J. W. Valle and C. Dive, Br. J. Cancer, 2012, 106, 508-516.
75. T. Nakamura, I. J. Fidler and K. R. Coombes, Cancer Res., 2007, 67, 139-148.
76. O. O. Ogunwobi, W. Puszyk, H.-J. Dong and C. Liu, PLoS One, 2013, 8, e63765.
77. L. Ren-Heidenreich, P. A. Davol, N. M. Kouttab, G. J. Elfenbein and L. G. Lum, Cancer, 2004, 100, 1095-1103.
78. G. Moldenhauer, A. V. Salnikov, S. Lüttgau, I. Herr, J. Anderl and H. Faulstich, J. Natl. Cancer Inst., 2012, 104, 622-634.
79. M. Lustberg, K. Jatana, M. Zborowski and J. Chalmers, in Minimal Residual Disease and Circulating Tumor Cells in Breast Cancer, eds. M. Ignatiadis, C. Sotiriou and K. Pantel, Springer Berlin Heidelberg, 2012, vol. 195, ch. 9, pp. 97-110.

1-30. (canceled)

31. A device for isolating a target cell from a population of cells, the device comprising:
a) one or more microfluidic channels, and
b) scaffolding particles conjugated with one or more ligands that bind to the target cell,
wherein the scaffolding particles with one or more ligands are attached to the surface of said one or more microfluidic channels.

32. The device of claim 31, wherein the scaffolding particles are attached to the surface of said one or more microfluidic channels by a spacer.

33. The device of claim 31, wherein the one or more microfluidic channels are quadrangular microfluidic channels.

34. The device of claim 33, wherein each of the microfluidic channels have a length of about 20-100 mm, a width of about 0.2-20 mm, and a height of about 20-200 μM.

35. The device of claim 31, wherein the scaffolding particles are nanoparticles.

36. The device of claim 35, wherein the nanoparticles are gold nanoparticles.

37. The device of claim 35, wherein the nanoparticles are attached to the surface of the microfluidic channels by a cleavable spacer.

38. The device of claim 37, wherein the cleavable spacer is a polymer.

39. The device of claim 38, wherein the polymer is a biocompatible polymer.

40. The device of claim 39. wherein the biocompatible polymer is polyethylene glycol.

41. The device of claim 31, wherein the one or more ligands are selected from the group consisting of DNA aptamers, RNA aptamers, XNA aptamers, peptide aptamers, antibodies, receptor binding proteins, and small molecule chemicals.

42. The device of claim 31, wherein the scaffolding particles are conjugated with a plurality of ligands.

43. The device of claim 42, wherein the plurality of ligands are selected from the group consisting of aptamers, antibodies, receptor-specific peptide ligands, receptor-specific hormone ligands and combinations thereof.

44. The device of claim 42, wherein the plurality of ligands comprises different DNA aptamers, said different DNA aptamers binding to different target sites on the surface of the target cell.

45. The device of claim 43, wherein the plurality of DNA aptamers comprises up to 95 different DNA aptamer sequences.

46. The device of claim 31, said device further comprising a micro-mixer.

47. The device of claim 46, wherein the micro-mixer is a herringbone groove-based micro-mixer.

48. The device of claim 31, further comprising one or more valves.

49. The device of claim 31, wherein said one or more ligands are selected from the aptamers of SEQ ID NOs: 1-26.

50. A method of isolating a target cell from a population of cells, the method comprising:
a) passing the population of cells through the device of claim 31 under conditions that permit the interaction and capture of the target cell by the scaffolding particle ligand conjugates within one or more microfluidic channels on said device,
b) passing a wash buffer through said one or more microfluidic channels to remove the cells non-specifically bound to the scaffolding particle ligand conjugates,
c) optionally, passing one or more reagents over to verify that the captured cells are truly target cells,
d) optionally, enumerating the cells captured,
e) releasing the captured target cell from the scaffolding particle ligand conjugates, and
f) collecting the released target cell.

51. The method of claim 50, wherein the population of cells originates from a tissue or body fluid of an organism.

52. The method of claim 51, wherein the tissue or body fluid is processed to prepare a sample containing the population of cells.

53. The method of claim 52, wherein the tissue is homogenized to prepare a slurry or solution containing the population of cells.

54. The method of claim 51, wherein the body fluid of the organism is blood.

55. The method of claim 54, wherein the blood is treated to lyse red blood cells (RBCs) found in said blood without damaging other cellular components found in said blood.

56. The method of claim 50, wherein the body fluid is blood from the organism.


SEQUENCE LISTING (26 sequences)

SEQ ID NO: 1 - DNA, 41 bp, Artificial Sequence
  Sgc8 aptamer sequence
atctaactgctgcgccgccgggaaaatactgtacggttaga

SEQ ID NO: 2 - DNA, 48 bp, Artificial Sequence
  TD05 aptamer sequence
aacaccgtggaggatagttcggtggctgttcagggtctcctcccggtg

SEQ ID NO: 3 - DNA, 51 bp, Artificial Sequence
  Sgc3B aptamer sequence
acttattcaattcctgtgggaaggctatagaggggccagtctatgaataag

SEQ ID NO: 4 - DNA, 88 bp, Artificial Sequence
  Sgd5 aptamer sequence
ataccagcttattcaattatcgtgggtcacagcagcggttgtgaggaagaaaggcggataacagataataagatagtaagtgcaatct

SEQ ID NO: 5 - DNA, 78 bp, Artificial Sequence
  KH2B05 aptamer sequence
atccagagtgacgcagcacacacaacctgctcataaactttactctgctcgaaccatctctggacacggtggcttagt

SEQ ID NO: 6 - DNA, 80 bp, Artificial Sequence
  KH1A02 aptamer sequence
atccagagtgacgcagcaggcatagatgtgcagctccaaggagaagaaggagttctgtgtattggacacggtggcttagt

SEQ ID NO: 7 - DNA, 78 bp, Artificial Sequence
  KH1C12 aptamer sequence
atccagagtgacgcagcatgccctagttactactactctttttagcaaacgccctcgctttggacacggtggcttagt

SEQ ID NO: 8 - DNA, 63 bp, Artificial Sequence
  TLS11a aptamer sequence
acagcatccccatgtgaacaatcgcattgtgattgttacggtttccgcctcatggacgtgctg

SEQ ID NO: 9 - DNA, 76 bp, Artificial Sequence
  PP3 aptamer sequence
atccagagtgacgcagcacgagccagacatctcacacctgttgcatatacattttgcatggacacggtggcttagt

SEQ ID NO: 10 - DNA, 62 bp, Artificial Sequence
  TV02 aptamer sequence
atcgtctgctccgtccaatacctgcatatacactttgcatgtggtttggtgtgaggtcgtgc

SEQ ID NO: 11 - DNA, 71 bp, Artificial Sequence
  HCH07 aptamer sequence
taccagtgcgatgctcaggccgatgtcaactttttctaactcactggttttgcctgacgcattcggttgac

SEQ ID NO: 12 - DNA, 59 bp, Artificial Sequence
  KDED2a-3 aptamer sequence
tgcccgcgaaaactgctattacgtgtgagaggaaagatcacgcgggttcgtggacacgg

SEQ ID NO: 13 - DNA, 81 bp, Artificial Sequence
  KCHA10 aptamer sequence
atccagagtgacgcagcaggggaggcgagagcgcacaataacgatggttgggacccaactgtttggacacggtggcttagt

SEQ ID NO: 14 - DNA, 45 bp, Artificial Sequence
  S11e aptamer sequence
atgcgaacaggtgggtgggttgggtggattgttcggcttcttgat

SEQ ID NO: 15 - DNA, 71 bp, Artificial Sequence
  DOV4 aptamer sequence
actcaacgaacgctgtggagggcatcagattaggatctataggttcggacatcgtgaggaccaggagagca

SEQ ID NO: 16 - DNA, 79 bp, Artificial Sequence
  aptTOV1 aptamer sequence
atccagagtgacgcagcagatctgtgtaggatcgcagtgtagtggacatttgatacgactggctcgacacggtggctta

SEQ ID NO: 17 - DNA, 76 bp, Artificial Sequence
  KMF2-1a aptamer sequence
aggcggcagtgtcagagtgaataggggatgtacaggtctgcacccactcgaggagtgactgagcgacgaagacccc

SEQ ID NO: 18 - DNA, 41 bp, Artificial Sequence
  EJ2 aptamer sequence
agtggtcgaactacacatccttgaactgcggaattatctac

SEQ ID NO: 19 - DNA, 75 bp, Artificial Sequence
  CSC01 aptamer sequence
accttggctgtcgtgttgtaggtggtttgctgcggtgggctcaagaagaaagcgcaaagtcagtggtcagagcgt

SEQ ID NO: 20 - DNA, 48 bp, Artificial Sequence
  Anti-EpCAM aptamer (SYL3C) sequence
cactacagaggttgcgtctgtcccacgttgtcatggggggttggcctg

SEQ ID NO: 21 - DNA, 117 bp, Artificial Sequence
  Anti-EGFR aptamer sequence
ggcgcuccgaccuuagucucugugccgcuauaaugcacggauuuaaucgccguagaaaagcaugucaaagccggaaccguguagcacagcagagaauuaaaugcccgccaugaccag

SEQ ID NO: 22 - DNA, 34 bp, Artificial Sequence
  Anti-PSMA aptamer sequence
accaagaccugacuucuaacuaagucuacguucc

SEQ ID NO: 23 - DNA, 51 bp, Artificial Sequence
  3′ biotinylated aptamer sequence
atctaactgctgcgccgccgggaaaatactgtacggttagatttttttttt

SEQ ID NO: 24 - DNA, 41 bp, Artificial Sequence; identical to SEQ ID NO: 1
  5′ pegylated and 3′ biotinylated aptamer sequence

SEQ ID NO: 25 - DNA, 58 bp, Artificial Sequence
  3′ biotinylated aptamer sequence
aacaccgtggaggatagttcggtggctgttcagggtctcctcccggtgtttttttttt

SEQ ID NO: 26 - DNA, 48 bp, Artificial Sequence; identical to SEQ ID NO: 2
  5′ pegylated and 3′ biotinylated aptamer sequence